(19) United States
(12) Patent Application Publication
(10) Pub. No.: US 2023/0382978 A1
(43) Pub. Date: Nov. 30, 2023

(54) ANTIBODY SPECIFIC FOR SARS-COV-2 RECEPTOR BINDING DOMAIN AND THERAPEUTIC METHODS

(71) Applicant: THE UNITED STATES OF AMERICA, AS REPRESENTED BY THE SECRETARY, DEPARTMENT OF HEALTH & HUMAN SERVICES, Bethesda, MD (US)

(72) Inventors: Kaitlyn Noelle SADTLER, Bethesda, MD (US); Matthew James MEMOLI, Bethesda, MD (US)

(21) Appl. No.: 18/031,814

(22) PCT Filed: Oct. 8, 2021
(86) PCT No.: PCT/US2021/054281

§ 371 Date: Apr. 13, 2023

Related U.S. Application Data
(60) Provisional application No. 63/225,248, filed on Jul. 23, 2021.
(60) Provisional application No. 63/092,350, filed on Oct. 15, 2020.

(51) Int. Cl. C07K 16/10

(52) U.S. Cl. CPC C07K 16/1003; C07K 2317/24; C07K 2317/30; C07K 2317/76; C07K 2317/92

(57) ABSTRACT

Antibodies against coronavirus receptor binding domain (SARS-CoV-2) are provided. The antibodies have neutralizing activity against coronaviruses and their activity. The anti-coronavirus receptor binding domain antibodies may be used in therapeutic treatment in patients with coronavirus infections and/or conditions associated with coronavirus infection.

### CROSS-REFERENCE TO RELATED APPLICATIONS

This is an International Application under the Patent Cooperation Treaty, claiming priority to U.S. Provisional Patent Application No. 63/092,350, filed 15 Oct. 2020, and claiming priority to U.S. Provisional Patent Application No. 63/225,248, filed 23 Jul. 2021, the contents of which are incorporated herein by reference in their entirety.

### REFERENCE TO SEQUENCE LISTING SUBMITTED ELECTRONICALLY

Pursuant to the EFS-Web legal framework and 37 CFR §§ 1.821-825 (see MPEP § 2442.03(a)), a Sequence Listing in the form of an ASCII-compliant text file (entitled “3000093-004977_Sequence_Listing_ST25.txt” created on 07 Oct. 2021 and 24,425 bytes in size) is submitted concurrently with the instant application, and the entire contents of the Sequence Listing are incorporated herein by reference.

### BACKGROUND

### 1. Field

The present disclosure relates to antibodies that specifically bind the SARS-CoV-2 receptor binding domain (COVID RBD). The antibodies have neutralizing activity against the action of SARS-CoV-2 virus and its biological function, and can be used as a therapeutic in patients presenting symptoms of coronavirus infection and complications thereof.

### 2. Description of Related Art

Viral infections are a continued problem for public health. In the 20^{th} and 21^{st} centuries, pandemics have been caused by novel viruses.

### Coronavirus

Coronavirus disease (COVID-19) is an infectious disease caused by a newly discovered coronavirus, Severe Acute Respiratory Syndrome CoronaVirus 2 (SARS-CoV-2). The SARS-CoV-2 was discovered in Wuhan Viral Pneumonia cases in late 2019, and was named by the World Health Organization on Jan. 12, 2020. It belongs to the beta genera of the Coronaviridae family identified in 2003, together with SARS coronavirus (SARS CoV), identified in 2003 and MERS coronavirus (MERS CoV) identified in 2012. The SARS-CoV-2 genome shares about 70% sequence identity with the SARS CoV virus and about 40% sequence similarity with the MERS CoV virus. WHO website (2020).

Most people infected with the COVID-19 virus will experience mild to moderate respiratory illness and recover without requiring special treatment. Older patients, e.g., over 60 years of age, and those with underlying medical problems such as cardiovascular disease, diabetes, chronic respiratory disease, and cancer, are more likely to develop serious illness. Centers for Disease Control website (2020). In susceptible populations, severe acute respiratory syndrome coronavirus 2 (SARS-CoV-2) may cause fatal human respiratory disease. Patients with SARS-CoV-2 often display the characteristics of acute lung injury (ALI), including diffuse alveolar damage (DAD), epithelial necrosis, and fibrin and hyaline deposition. Many patients who die of SARS-CoV-2 develop acute respiratory distress syndrome (ARDS), a severe form of acute lung injury. Li & Ma Critical Care (2020) 24: 198. Outbreaks of severe acute respiratory infections of emerging viruses, including Middle Eastern respiratory syndrome CoVs (MERS-CoV) and the novel coronavirus (SARS-CoV-2, also referred to as “COVID-19”) show a need in the art for effective agents for the treatment and/or prevention of coronavirus infections.

### BRIEF SUMMARY

The present invention encompasses antibodies (including antigen-binding molecules comprising, or alternatively consisting of, antibody fragments or variants thereof) that immunospecifically bind to a polypeptide or polypeptide fragment of the SARS-CoV-2 receptor binding domain (COVID RBD), compositions, and methods of use thereof. In particular, the invention provides antibodies (including antigen-binding molecules comprising, or alternatively consisting of, antibody fragments or variants thereof) that immunospecifically bind to a polypeptide or polypeptide fragment of the receptor binding domain (RBD) of a coronavirus (SEQ ID NO: 2 and/or SEQ ID NO: 3), preferably the receptor binding domain of SARS-CoV-2 (SEQ ID NO: 1 and/or SEQ ID NO: 4). The present invention further provides methods and compositions for preventing, treating or ameliorating diseases or disorders associated with coronavirus infection, preferably SARS-CoV-2 (COVID-19) in an animal, preferably a mammal, and most preferably a human, comprising, or alternatively consisting of, administering to said animal an effective amount of one or more antibodies (including antigen-binding molecules comprising, or alternatively consisting of, antibody fragments or variants thereof) that immunospecifically bind to a coronavirus RBD. Diseases and disorders which can be prevented, treated or ameliorated by administering an effective amount of an antibody described herein include, but are not limited to, coronavirus infections, including but not limited to a SARS-CoV, MERS-CoV, SARS-CoV-2 (COVID-19) virus, or a combination thereof.

The present inventors identified antibodies that immunospecifically bind to coronavirus RBD, in particular, to the coronavirus RBD, including the full spike ectodomain trimer. The antibodies described herein have a general “Y” structure consisting of four polypeptides, two heavy chains and two light chains. Each heavy and light chain consists of a constant region and a variable region, e.g., variable heavy (HV) and variable light (LH). Each heavy and light chain comprises a set of three (3) complementarity-determining regions (CDRs) in the variable regions. Antigen-binding molecules comprising, or alternatively consisting of, fragments or variants of these antibodies (e.g., including VH domains, VH CDRs, VL domains, or VL CDRs having an amino acid sequence of any one of those described herein), that immunospecifically bind to coronavirus RBD (SEQ ID NO: 1, 2, 3, 4, or a combination thereof), are also encompassed by the invention, as are nucleic acid molecules that encode these antibodies, vectors and host cells comprising the same, and/or antigen-binding molecules.

In one embodiment, antibodies may comprise a heavy chain constant region comprising an amino acid sequence of SEQ ID NO: 5. Antigen-binding molecules comprising, or alternatively consisting of, fragments or variants of these monoclonal antibodies (e.g., including VH domains, VH CDRs, VL domains, or VL CDRs), that immunospecifically bind to coronavirus RBD, also are described are nucleic acid molecules that encode these antibodies, vectors and host cells comprising the same, and/or antigen-binding molecules.

In one embodiment, antibodies may comprise a light chain constant region comprising an amino acid sequence of SEQ ID NO: 6. Antigen-binding molecules comprising, or alternatively consisting of, fragments or variants of these monoclonal antibodies (e.g., including VH domains, VH CDRs, VL domains, or VL CDRs), that immunospecifically bind to a coronavirus RBD, also are described are nucleic acid molecules that encode these antibodies, vectors and host cells comprising the same, and/or antigen-binding molecules.

In an embodiment, antibodies may comprise a VH domain comprising an amino acid sequence of SEQ ID NO: 24, and a VL domain comprising an amino acid sequence of SEQ ID NO: 23. Antigen-binding molecules comprising, or alternatively consisting of, fragments or variants of these monoclonal antibodies (e.g., including VH domains, VH CDRs, VL domains, or VL CDRs having an amino acid sequence of any one of those described herein), that immunospecifically bind to coronavirus RBD, are also encompassed by the invention, as are nucleic acid molecules that encode these antibodies, vectors and host cells comprising the same, and/or antigen-binding molecules.

In an embodiment, the antibodies described herein comprise a VH domain comprising, or alternatively consisting of, a polypeptide sequence selected from the group consisting of SEQ ID NO: 16, 18, and 22 and a VL domain comprising, or alternatively consisting of, a polypeptide sequence selected from the group consisting of SEQ ID NO: 9, 11, and 13. In an embodiment, antibodies of the present invention comprise a VH domain comprising, or alternatively consisting of, the polypeptide of SEQ ID NO: 24 and a VL domain comprising, or alternatively consisting of, the polypeptide of SEQ ID NO: 23.

Molecules comprising, or alternatively consisting of, fragments or variants of VH domains having an amino acid sequence of SEQ ID NO: 24 (including VH CDRs), and VL domains having an amino acid sequence of SEQ ID NO: 23 (including VL CDRs), that immunospecifically bind to a coronavirus RBD, are also encompassed by the invention, as are nucleic acid molecules that encode these antigen-binding molecules.

In one embodiment, antibodies (including molecules comprising, or alternatively consisting of, antibody fragments or variants thereof) may immunospecifically bind to a polypeptide or a polypeptide fragment of a coronavirus RBD, said antibodies comprising, or alternatively consisting of, a polypeptide having the amino acid sequence of any one, two, three or more of the VH complementarity determining regions (“CDRs”) (e.g., VH CDR1, VH CDR2, or VH CDR3) of a VH domain having an amino acid sequence of SEQ ID NO: 24 and/or any one, two, three or more of the VL CDRs (e.g., VL CDR1, VL CDR2, or VL CDR3) of a VL domain having an amino acid sequence of SEQ ID NO: 23. In one embodiment, antibodies of the present invention comprise a polypeptide having the amino acid sequence of any one of the VH CDR1s of a VH domain having an amino acid sequence of SEQ ID NO: 17 and/or any one of the VL CDR1s of a VL domain having an amino acid sequence of SEQ ID NO: 10. In many embodiments, antibodies of the present invention comprise a polypeptide having the amino acid sequence of any one of the VH CDR2s of a VH domain having an amino acid sequence of SEQ ID NO: 19 and/or any one of the VL CDR2s of a VL domain having an amino acid sequence of SEQ ID NO: 12. In several embodiments, antibodies of the present invention comprise a polypeptide having the amino acid sequence of any one of the VH CDR3s of a VH domain having an amino acid sequence of SEQ ID NO: 21 and/or any one of the VL CDR3s of a VL domain having an amino acid sequence of SEQ ID NO: 14. Molecules comprising, or alternatively consisting of, fragments or variants of the VH domains of SEQ ID NO: 17,19,21, (e.g., VH CDRs), and/or the VL domains of SEQ ID NO: 10,12,14 (e.g., VL CDRs), that immunospecifically bind to a coronavirus RBD, are also encompassed by the invention, as are nucleic acid molecules that encode these antibodies, and/or molecules.

In an embodiment, antibodies of the present invention (including molecules comprising, or alternatively consisting of, antibody fragments or variants thereof) immunospecifically bind to a polypeptide or polypeptide fragment of a coronavirus RBD, and comprise, or alternatively consist of, a polypeptide having the amino acid sequence of any one of the VH CDR1s of the VH domains of SEQ ID NO: 17, any one of the VH CDR2s of the VH domains of SEQ ID NO: 19, and/or any one of the VH CDR3s of the VH domains of SEQ ID NO: 21. In another embodiment, antibodies of the present invention comprise, or alternatively consist of, a polypeptide having the amino acid sequence of any one of the VL CDR1s of the VL domains of SEQ ID NO: 10, any one of the VL CDR2s of the VL domains of SEQ ID NO: 12, and/or any one of the VL CDR3s of the VL domains of SEQ ID NO: 14. In an embodiment, antibodies of the present invention comprise CDR1, CDR2, and CDR3 of the VH domain of SEQ ID NO: 24 and/or CDR1, CDR2, and CDR3 of the VL domain of SEQ ID NO: 23. Molecules comprising, or alternatively consisting of, fragments or variants of these antibodies (e.g., including VH domains, VH CDRs, VL domains, or VL CDRs having an amino acid sequence within any one of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and combinations of the amino acid sequences of SEQ ID NOs: 7-24), that immunospecifically bind to a coronavirus RBD, are also encompassed by the invention, as are nucleic acid molecules that encode these antibodies, and/or molecules.

In one embodiment, antibodies (including molecules comprising, or alternatively consisting of, antibody fragments or variants thereof) may immunospecifically bind to the receptor binding domain of a coronavirus, for example, amino acids 331-524 of the Spike protein of SARS-CoV-2 (SEQ ID NO: 1), amino acid residues 318-510 of the Spike protein of SARS-CoV (SEQ ID NO: 2), amino acid residues 377-588 of the MERS-CoV spike protein (SEQ ID NO: 3), amino acid residues 318-510 of the Spike protein of SARS-CoV, amino acid residues 377-588 of the MERS-CoV spike protein; amino acids 319-541 of the SARS CoV-2 spike receptor-binding domain (SEQ ID NO: 4).

In an embodiment, antibodies of the present invention immunospecifically bind to a coronavirus, for example, a polypeptide comprising, or alternative consisting of, amino acids 331-524 of the Spike protein of SARS-CoV-2 (SEQ ID NO: 1), amino acid residues 318-510 of the Spike protein of SARS-CoV (SEQ ID NO: 2), amino acid residues 377-588 of the MERS-CoV spike protein (SEQ ID NO: 3), amino acid residues 318-510 of the Spike protein of SARS-CoV, amino acid residues 377-588 of the MERS-CoV spike protein; amino acids 319-541 of the SARS CoV-2 spike receptor-binding domain (SEQ ID NO: 4) and comprise, or alternatively consist of, a VH domain, VH CDR1, VH CDR2, VH CDR3, VL domain, VL CDR1, VL CDR2, and/or VL CDR3 corresponding to one or more VH domains of SEQ ID NO: 24 and/or one or more VL domains of SEQ ID NO: 23. In several embodiments, antibodies of the present invention immunospecifically bind to a coronavirus (e.g., a polypeptide comprising, or alternative consisting of, the amino acid sequence of SEQ ID NO: 1, 2, 3, and/or 4) and comprise, or alternatively consist of, a VH domain (SEQ ID NO: 24), VH CDR1 (SEQ ID NO: 17), VH CDR2 (SEQ ID NO: 19), VH CDR3 (SEQ ID NO: 21), VL domain (SEQ ID NO: 23), VL CDR1 (SEQ ID NO: 10), VL CDR2 (SEQ ID NO: 12), and/or VL CDR3 (SEQ ID NO: 14) corresponding to one or more VH domains of SEQ ID NO: 24 and/or one or more VL domains of SEQ ID NO: 23.

Molecules comprising, or alternatively consisting of, fragments or variants the heavy chain constant region having an amino acid sequence of SEQ ID NO: 5 that immunospecifically bind to a coronavirus RBD, are also encompassed by the invention, as are nucleic acid molecules that encode these antigen-binding molecules.

Molecules comprising, or alternatively consisting of, fragments or variants the light chain constant region having an amino acid sequence of SEQ ID NO: 6 that immunospecifically bind to a coronavirus RBD, are also encompassed by the invention, as are nucleic acid molecules that encode these antigen-binding molecules.

In an embodiment, antibodies (including molecules comprising, or alternatively consisting of, antibody fragments or variants thereof) may immunospecifically bind to a polypeptide or a polypeptide fragment of a coronavirus RBD (SEQ ID NO: 1, 2, 3, 4, or combinations thereof), said antibodies comprising, or alternatively consisting of, a polypeptide having the amino acid sequence of any one, two, three or more of the VH complementarity determining regions (“CDRs”) (e.g., VH CDR1, VH CDR2, or VH CDR3) of a VH domain and/or any one, two, three or more of the VL CDRs (e.g., VL CDR1, VL CDR2, or VL CDR3) of a VL domain.

In another embodiment, antibodies of the present invention (including molecules comprising, or alternatively consisting of, antibody fragments or variants thereof) immunospecifically bind to a polypeptide or polypeptide fragment of a coronavirus RBD, and comprise, or alternatively consist of, a polypeptide having the amino acid sequence of the Heavy Chain constant region of the amino acid sequence of SEQ ID NO: 5 and of the Light Chain constant region of the amino acid sequence of SEQ ID NO: 6.

A VH domain of an amino acid sequence disclosed herein may be combined with a VL domain of an amino acid sequence disclosed herein, or other VL domains, to provide a VH/VL pairing representing an antigen-binding site of an antibody. Similarly, a VL domain of an amino acid sequence disclosed herein may be combined with a VH domain of an amino acid sequence disclosed herein, or other VH domains. Further, one or more CDRs disclosed herein may be taken from a VH or VL domain and incorporated into a suitable framework as discussed infra.

In one embodiment, antibodies (including molecules comprising, or alternatively consisting of, antibody fragments or variants thereof (including derivatives)) may comprise, or alternatively consist of, of VH domains, VL domains and/or CDRs described herein, which antibodies, immunospecifically bind to coronavirus receptor binding domain (RBD) (e.g., SEQ ID NO: 1, 2, 3, 4) and can be routinely assayed for immunospecific binding to coronavirus RBD using methods known in the art, such as, for example, the immunoassays disclosed herein. Antibodies and antibody fragments or variants (including derivatives) described herein may include, for example, one or more amino acid sequence alterations (addition, deletion, substitution and/or insertion of an amino acid residue). These alterations may be made in one or more framework regions and/or one or more CDRs. Amino acid changes in the CDRs may be preferably performed without disrupting the coronavirus RBD binding specificity. The antibodies described herein (including antibody fragments, and variants and derivative thereof) can be routinely made by methods known in the art. Molecules comprising, or alternatively consisting of, fragments or variants of any of the VH domains, VH CDRs, VL domains, and VL CDRs whose sequences are specifically disclosed herein may be employed in accordance with the present invention. Nucleic acid molecules encoding these antibodies and molecules (including fragments, variants, and derivatives) are also encompassed by the invention.

In an embodiment, the heavy chain constant region may comprise an amino acid sequence that shares at least about 80% sequence homology to the amino acid sequence of SEQ ID NO: 5.

In an embodiment, the light chain constant region may comprise an amino acid sequence that shares at least about 80% sequence homology to the amino acid sequence of SEQ ID NO: 6.

In an embodiment, the light chain may comprise an amino acid sequence that shares at least about 80% sequence homology to the amino acid sequence of SEQ ID NO: 7.

In an embodiment, the heavy chain may comprise an amino acid sequence that shares at least about 80% sequence homology to the amino acid sequence of SEQ ID NO: 8.

In an embodiment, the light chain variable region may comprise an amino acid sequence that shares at least about 80% sequence homology to the amino acid sequence of SEQ ID NO: 23.

In an embodiment, the heavy chain variable region may comprise an amino acid sequence that shares at least about 80% sequence homology to the amino acid sequence of SEQ ID NO: 24.

In an embodiment, the light chain variable region may comprise CDR1 comprising an amino acid sequence of SEQ ID NO: 10, CDR2 comprising an amino acid sequence of SEQ ID NO: 12, and CDR3 comprising an amino acid sequence of SEQ ID NO: 14.

In an embodiment, the heavy chain variable region may comprise CDR1 comprising an amino acid sequence of SEQ ID NO: 17, CDR2 comprising an amino acid sequence of SEQ ID NO: 19, and CDR3 comprising an amino acid sequence of SEQ ID NO: 21.

In an embodiment, the light chain variable region may comprise framework sections comprising the amino acid sequences of SEQ ID NOs: 9, 11, 13, and 15.

In an embodiment, the heavy chain variable region may comprise framework sections comprising the amino acid sequences of SEQ ID NOs: 16, 18, 20, and 22.

In any embodiment, the sequence homology may be at least about 85%, 90%, 91%, 92%, 93%, 94%, 95%, 96%, 97%, 98%, 99%, or 100% sequence homology.

In one embodiment, panels of antibodies (including molecules comprising, or alternatively consisting of, antibody fragments or variants) are described, wherein the panel members correspond to one, two, three, four, five, ten, fifteen, twenty, or more different antibodies described herein (e.g., whole antibodies, Fabs, F(ab′) 2 fragments, Fd fragments, disulfide-linked Fvs (sdFvs), antiidiotypic (anti-Id) antibodies, and scFvs). The present invention further provides mixtures of antibodies, wherein the mixture corresponds to one, two, three, four, five, ten, fifteen, twenty, or more different antibodies described herein (e.g., whole antibodies, Fabs, F(ab′) 2 fragments, Fd fragments, disulfide-linked Fvs (sdFvs), antiidiotypic (anti-Id) antibodies, and scFvs).

In one embodiment, compositions may comprise or consist of one, two, three, four, five, ten, fifteen, twenty, or more antibodies of the present invention (including molecules comprising, or alternatively consisting of, antibody fragments or variants thereof). A composition described herein may comprise, or alternatively consist of, one, two, three, four, five, ten, fifteen, twenty, or more amino acid sequences of one or more antibodies or fragments or variants thereof. Alternatively, a composition described herein may comprise, or alternatively consist of, nucleic acid molecules encoding one or more antibodies described herein.

In one embodiment, fusion proteins may comprise an antibody (including molecules comprising, or alternatively consisting of, antibody fragments or variants thereof) described herein, and a heterologous polypeptide (e.g., a polypeptide unrelated to an antibody or antibody domain). Nucleic acid molecules encoding these fusion proteins are also encompassed by the invention. A composition of the present invention may comprise, or alternatively consist of, one, two, three, four, five, ten, fifteen, twenty or more fusion proteins described herein. Alternatively, a composition described herein may comprise, or alternatively consist of, nucleic acid molecules encoding one, two, three, four, five, ten, fifteen, twenty or more fusion proteins described herein.

In one embodiment, a recombinant nucleic acid molecule, generally isolated, encoding an antibody (including molecules which may comprise or consist of an antibody fragment or variant thereof) described herein. The present invention also provides a host cell transformed with a nucleic acid molecule described herein and progeny thereof. The present invention also provides a method for the production of an antibody (including a molecule comprising, or alternatively consisting of, an antibody fragment or variant thereof) described herein. The present invention further provides a method of expressing an antibody (including a molecule comprising, or alternatively consisting of, an antibody fragment or variant thereof) described herein from a recombinant nucleic acid molecule. These and other aspects described herein are described in further detail below.

In one embodiment, methods and compositions for treating coronavirus infections, preferably SARS-CoV-2 (COVID-19) infection in an animal, preferably a mammal, and most preferably a human, may comprise using antibodies (including molecules which comprise, or alternatively consist of, antibody fragments or variants thereof) that immunospecifically bind to a coronavirus RBD. Diseases and disorders which can be treating with the antibodies described herein include, but are not limited to acute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIG. 1 depicts sequence alignment of RBDs of SARS-CoV-2 (SEQ ID NO: 1), SARS-CoV (SEQ ID NO: 2), and MERS-CoV (SEQ ID NO: 3) spike (S) proteins. GenBank accession numbers are QHR63250.1 (SARS-CoV-2 S), AY278488.2 (SARS-CoV S), and AFS88936.1 (MERS-CoV S). Variable amino acid residues between SARS-CoV-2 and SARS-CoV are indicated shaded and bold (e.g., R). Conserved residues among SARS-CoV-2, SARS-CoV, and MERS-CoV are indicated as follows: asterisks (*) represent fully conserved residues, colons (:) represent highly conserved residues, and periods (.) represent lowly conserved residues. The alignment was performed using Clustal Omega. Reproduced from Tai et al. Cellular & Molecular Immunology (2020) 17: 613-620.

FIG. 2 depicts a proposed use of the antibodies and antigen-binding fragments described herein for a SARS-CoV-2 therapeutic.

FIG. 3 depicts the specific binding of the antibodies and antigen-binding fragments described herein against RBD and the isolated receptor binding domain (RBD) construct and a full spike ectodomain trimer of the SARS-CoV-2 virus. Standard Curve of Blood serum spiked with IgA RBD recombinant monoclonal IgA antibody at a range of recombinant antibody concentrations. Added into ELISA at 1:400 dilution.

FIG. 4 depicts the amino acid sequence of a variable region of a light chain (SEQ ID NO: 23) from an exemplary antibody described herein. CDR sequences are annotated with Chothia scheme.

FIG. 5 depicts an amino acid sequence of the variable region of a heavy chain (SEQ ID NO: 24) from an exemplary antibody described herein. CDR sequences are annotated with Chothia scheme.

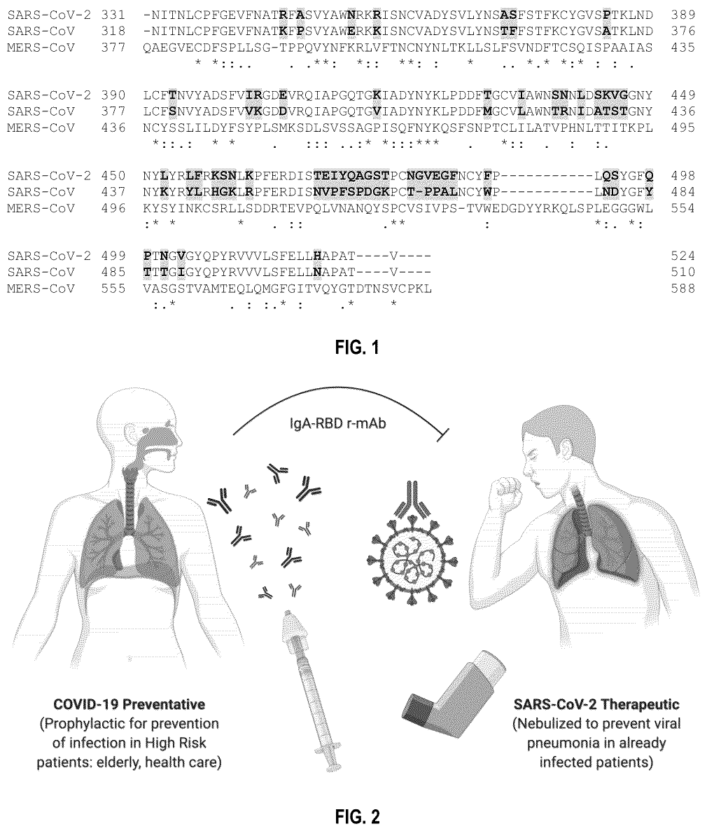

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

### Definitions

As used herein, the term “anti-coronavirus RBD antibody,” refers broadly to an antibody that is capable of binding to receptor binding domain of a coronavirus, preferably a SARS-CoV-2 receptor binding domain (COVID RBD). Further, “anti-coronavirus RBD antibody,” refers broadly to an antibody that is capable of binding to receptor binding domain of a coronavirus, preferably a SARS-CoV-2 receptor binding domain (COVID RBD), for example SARS-CoV, MERS-CoV, SARS-CoV-2 (COVID-19) virus, or a combination thereof.

As used herein, an anti-coronavirus RBD antibody has the property binding to a coronavirus RBD such that the coronavirus, preferably SARS-CoV-2 virus, is inactivated (“neutralized”). An anti-coronavirus RBD antibody candidate can be tested for such activity, for example, by adsorbing anti-coronavirus RBD antibody to immobilized coronavirus RBD followed by subjecting the adsorbed antibody to elution with an excess of isolated RBD polypeptides. If an eluent comprising an excess of the selected RBD produces an eluate containing a greater concentration of the candidate antibody than the concentration of candidate antibody present in an eluate produced by a “blank” eluent (the same eluent containing no RBD) in a control elution, as determined by, e.g., radioimmunoassays performed on the respective eluates with radiolabelled, soluble coronavirus RBD, then the candidate antibody competes with the selected anti-coronavirus RBD antibody for binding to a coronavirus RBD.

As used herein, an anti-coronavirus RBD antibody with the property or capability of “neutralizing SARS-CoV-2,” refers broadly to as an anti-coronavirus RBD antibody capable of reducing or inhibiting the activity of coronaviruses, preferably SARS-CoV-2. An anti-coronavirus RBD antibody candidate can be tested for such activity, for example, by measuring prevention of SARS-CoV-2 infection and/or activity in one or more biological assays. The anti-coronavirus RBD antibodies may bind an epitope contained with the amino acid sequences of SEQ ID NOS: 1, 2, 3, 4, or a combination thereof.

As used herein, “COVID-19”, “COVID”, and “SARS-CoV-2, all refer to the SARS-CoV-2 virus, the coronavirus that causes coronavirus disease 2019, also referred to as COVID-19.

As used herein, “coronavirus,” refers broadly to viruses that are members of the coronavirus group. Coronaviruses are named for the crown-like spikes on their surface. There are four main sub-groupings of coronaviruses, known as alpha, beta, gamma, and delta. Preferred coronaviruses include but are not limited to SARS-CoV-1, MERS-CoV, SARS-CoV-2 (COVID-19), HCoV-HCoV-HKU1, HCoV-NL63, HCoV-229E or a combination thereof.

“Substantially free,” as used herein, refers broadly to the presence of a specific component in an amount less than 1%, preferably less than 0.1% or 0.01%. More preferably, the term “substantially free” refers broadly to the presence of a specific component in an amount less than 0.001% The amount may be expressed as w/w or w/v depending on the composition.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Antibodies are heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Chothia et al. J. Mol. Biol. 186:651 (1985); Novotny and Haber, Proc. Natl. Acad. Sci. U.S.A. 82:4592 (1985)).

The term “variable” refers broadly to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al. Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′) 2 fragment that has two antigen-combining sites and is still capable of cross-linking antigen.

“Fv” is the minimum antibody fragment which contains a complete antigen-recognition and binding site. In a two-chain Fv species, this region consists of a dimer of one heavy and one light chain variable domain in tight, non-covalent association. In a single-chain Fv species, one heavy and one light chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′) 2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG 1, IgG2, IgG3, IgG4, IgA1, and IgA2. The heavy-chain constant domains that correspond to the different classes of immunoglobulins are called α, δ, ε, γ, and μ, respectively. In addition, secretory immunoglobulin A (IgA), the predominant immunoglobulin isotype present in airway secretions, is composed of two IgA molecules (dimeric IgA), a joining protein (J chain), and a secretory component. The subunit structures and three-dimensional configurations of different classes of immunoglobulins are well known. “Therapeutic Antibody Engineering” (1^{st} Ed.) Strohl & Strohl Woodhead Publishing (2012). The term “antibody” specifically encompasses monoclonal antibodies, including antibody fragment clones. The anti-coronavirus RBD antibodies described herein comprise CDRs specifically bind to an epitope in the amino acid sequences of SEQ ID NOS: 1, 2, 3, 4, or a combination thereof, preferably the amino acid sequence of SEQ ID NO: 4. The antibodies of the invention preferably bind to an epitope consisting of 6 to 12 residues within the receptor binding domain of amino acids 319-541 of the SARS CoV-2 spike receptor-binding domain (SEQ ID NO: 4).

“Antibody fragments” comprise a portion of an intact antibody, generally the antigen binding or variable region of the intact antibody. Examples of antibody fragments include Fab, Fab′, F(ab′) 2, and Fv fragments; diabodies; single-chain antibody molecules, including single-chain Fv (scFv) molecules; and multispecific antibodies formed from antibody fragments. “Human Monoclonal Antibodies: Methods and Protocols” (2^{nd} Ed.) Steinitz (Ed.) Humana Press (2019).

The term “monoclonal antibody” as used herein refers to an antibody (or antibody fragment) obtained from a population of substantially homogeneous antibodies, e.g., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al. Nature 256:495 (1975), or may be made by recombinant DNA methods (See, e.g., U.S. Pat. No. 4,816,567). The “monoclonal antibodies” also include clones of antigen-recognition and binding-site containing antibody fragments (Fv clones) isolated from phage antibody libraries using the techniques described in Clackson, et al. Nature, 352:624-628 (1991) and Marks et al. J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567 to Cabilly et al.; Morrison et al. Proc. Natl. Acad. Sci. USA, 81:6851-6855 (1984)); “Antibody Engineering” Volume 2 (2^{nd} Ed.) Kontermann & Dübe'. Springer Press (2010).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody.

“Humanized” forms of non-human (e.g., murine) antibodies are chimeric immunoglobulins, immunoglobulin chains or fragments thereof (such as Fv, Fab, Fab′, F(ab′) 2 or other antigen-binding subsequences of antibodies) which contain minimal sequence derived from non-human immunoglobulin. For the most part, humanized antibodies are human immunoglobulins (recipient antibody) in which residues from a complementarity-determining region (CDR) of the recipient are replaced by residues from a CDR of a non-human species (such as mouse, rat or rabbit) or a synthetic sequence (donor antibody), having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In an embodiment, all the CDRs are from the donor immunoglobulin in a humanized immunoglobulin. Constant regions need not be present, but if they are, they should be substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering. See for example, U.S. Pat. No. 5,585,089.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. Proc. Natl. Acad. Sci. USA 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “variant” as used herein refers broadly to a polypeptide that possesses a similar or identical function as a coronavirus RBD polypeptide, an anti-coronavirus RBD antibody or antibody fragment thereof, but does not necessarily comprise a similar or identical amino acid sequence of a coronavirus RBD polypeptide, anti-coronavirus RBD or antibody fragment thereof, or possess a similar or identical structure of a coronavirus RBD polypeptide, an anti-coronavirus RBD antibody or antibody fragment thereof. A variant having a similar amino acid identity refers to a polypeptide that satisfies at least one of the following: (a) a polypeptide comprising, or alternatively consisting of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identical to the amino acid sequence of a coronavirus RBD polypeptide, anti-coronavirus RBD or antibody fragment thereof (comprising a VH domain (SEQ ID NO: 24), VHCDRs (SEQ ID NOs: 17, 19, 21), VL domain (SEQ ID NO: 23), or VLCDR (SEQ ID NOs: 10, 12, 14)); (b) a polypeptide encoded by a nucleotide sequence, the complementary sequence of which hybridizes under stringent conditions to a nucleotide sequence encoding a coronavirus RBD polypeptide or fragment thereof, an anti-coronavirus RBD antibody or antibody fragment thereof (comprising a VH domain (SEQ ID NO: 24), VHCDRs (SEQ ID NOs: 17, 19, 21), VL domain (SEQ ID NO: 23), or VLCDR (SEQ ID NOs: 10, 12, 14)), of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, identical to the nucleotide sequence encoding a coronavirus RBD polypeptide or fragment thereof, an anti-coronavirus RBD antibody or antibody fragment thereof (comprising a VH domain (SEQ ID NO: 24), VHCDRs (SEQ ID NOs: 17, 19, 21), VL domain (SEQ ID NO: 23), or VLCDR (SEQ ID NOs: 10, 12, 14)). A polypeptide with similar structure to a coronavirus RBD polypeptide or fragment thereof, an anti-coronavirus RBD antibody or antibody fragment thereof, described herein refers to a polypeptide that has a similar secondary, tertiary or quaternary structure of a coronavirus RBD polypeptide or fragment thereof, an anti-coronavirus RBD antibody, or antibody fragment thereof, described herein. The structure of a polypeptide can be determined by methods known to those skilled in the art, including but not limited to, X-ray crystallography, nuclear magnetic resonance, and crystallographic electron microscopy. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. The percent identity between the two sequences is a function of the number of identical positions shared by the sequences (e.g., % identity=number of identical overlapping positions/total number of positions×100%). In one embodiment, the two sequences are the same length.

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA 87:2264-2268 (1990), modified as in Karlin and Altschul Proc. Natl. Acad. Sci. USA 90:5873-5877 (1993). The BLASTn and BLASTx programs of Altschul, et al. J. Mal. Biol. 215:403-410(1990) have incorporated such an algorithm. BLAST nucleotide searches can be performed with the BLASTn program, score=100, wordlength=12 to obtain nucleotide sequences homologous to a nucleic acid molecule described herein. BLAST protein searches can be performed with the BLASTx program, score=50, wordlength=3 to obtain amino acid sequences homologous to a protein molecule described herein. To obtain gapped alignments for comparison purposes, Gapped BLAST can be utilized as described in Altschul et al. Nucleic Acids Res. 25:3389-3402(1997). Alternatively, PSI-BLAST can be used to perform an iterated search which detects distant relationships between molecules (Id.). When utilizing BLAST, Gapped BLAST, and PSI-BLAST programs, the default parameters of the respective programs (e.g., BLASTx and BLASTn) can be used. (See ncbi.nlm.nih.gov).

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci., 10 :3-5(1994); and FASTA described in Pearson and Lipman Proc. Natl. Acad. Sci. 85:2444-8(1988). Within FASTA, ktup is a control option that sets the sensitivity and speed of the search.

“Conservative” amino acid substitutions are those substitutions that do not substantially affect or decrease the affinity of a protein, such as an antibody to a coronavirus RBD. For example, a monoclonal antibody that immunospecifically binds a coronavirus RBD can include at most about 1, at most about 2, at most about 5, at most about 10, or at most about 15 conservative substitutions and immunospecifically bind a coronavirus RBD polypeptide. The term “conservative variant” also includes the use of a substituted amino acid in place of an unsubstituted parent amino acid, provided that antibody immunospecifically binds a coronavirus RBD. Non-conservative substitutions are those that reduce an activity or binding to a coronavirus RBD.

Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “derivative” as used herein, refers to a variant polypeptide described herein that comprises, or alternatively consists of, an amino acid sequence of a coronavirus RBD polypeptide or fragment thereof, or an antibody described herein that immunospecifically binds to a coronavirus RBD, which has been altered by the introduction of amino acid residue substitutions, deletions or additions. The term “derivative” as used herein also refers to a coronavirus RBD polypeptide or fragment thereof, an antibody that immunospecifically binds to a coronavirus RBD which has been modified, e.g., by the covalent attachment of any type of molecule to the polypeptide. For example, but not by way of limitation a coronavirus RBD polypeptide or fragment thereof, or an anti-coronavirus RBD antibody, may be modified, e.g., by glycosylation, acetylation, pegylation, phosphorylation, amidation, derivatization by known protecting/blocking groups, proteolytic cleavage, linkage to a cellular ligand or other protein, etc. A derivative of a coronavirus RBD polypeptide or fragment thereof, or an anti-coronavirus RBD antibody or fragment thereof, may be modified by chemical modifications using techniques known to those of skill in the art, including, but not limited to, specific chemical cleavage, acetylation, formylation, metabolic synthesis of tunicamycin, etc. Further, a derivative of a coronavirus RBD polypeptide or fragment thereof, or an anti-coronavirus RBD antibody or fragment thereof, may contain one or more non-classical amino acids. A polypeptide derivative possesses a similar or identical function as a coronavirus RBD polypeptide or fragment thereof, or an anti-coronavirus RBD antibody or fragment thereof, described herein.

The term “epitopes” as used herein refers to portions of a coronavirus RBD having antigenic or immunogenic activity in an animal, preferably a mammal. An epitope having immunogenic activity is a portion of a coronavirus RBD that elicits an antibody response in an animal. An epitope having antigenic activity is a portion of a coronavirus RBD to which an antibody immunospecifically binds as determined by any method known in the art, for example, by the immunoassays described herein. Antigenic epitopes need not necessarily be immunogenic.

The term “fragment” as used herein refers broadly to a polypeptide comprising an amino acid sequence of at least 5 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues, at least 45 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues, of the amino acid sequence of a coronavirus RBD, or an anti-coronavirus RBD antibody (including molecules such as scFvs, that comprise, or alternatively consist of, antibody fragments or variants thereof) that immunospecifically binds to a coronavirus RBD comprising the amino acid sequence of SEQ ID NOS: 1, 2, 3, 4, or a combination thereof, preferably the amino acid sequence of SEQ ID NO: 4.

The term “fusion protein” as used herein refers broadly to a polypeptide that comprises, or alternatively consists of, an amino acid sequence of an anti-coronavirus RBD antibody described herein and an amino acid sequence of a heterologous polypeptide (e.g., a polypeptide unrelated to an antibody or antibody domain).

The term “host cell” as used herein refers broadly to the particular subject cell transfected with a nucleic acid molecule and the progeny or potential progeny of such a cell. Progeny may not be identical to the parent cell transfected with the nucleic acid molecule due to mutations or environmental influences that may occur in succeeding generations or integration of the nucleic acid molecule into the host cell genome.

“Treatment” refers broadly to both therapeutic treatment and prophylactic or preventative measures. Those in need of treatment include those already with the disorder as well as those in which the disorder is to be prevented. As used herein, the term “treating,” refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms. Therapy also encompasses “prophylaxis”. The term “reduced”, for purpose of therapy, refers broadly to the clinically significant reduction in signs and/or symptoms. Therapy includes treating relapses or recurrent signs and/or symptoms. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms.

“Effective amount,” as used herein, refers broadly to the amount of a compound, antibody, antigen, or cells that, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this invention.

“Mammal,” as used herein, refers broadly to any and all warm-blooded vertebrate animals of the class Mammalia, characterized by a covering of hair on the skin and, in the female, milk-producing mammary glands for nourishing the young. Mammals include, but are not limited to, humans, domestic and farm animals, and zoo, sports, or pet animals. Examples of mammals include but are not limited to alpacas, armadillos, capybaras, cats, camels, chimpanzees, chinchillas, cattle, dogs, gerbils, goats, gorillas, guinea pigs, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C. Similarly, the term “subject” or “patient” includes both human and veterinary subjects and/or patients.

### Coronavirus

Coronaviruses are RNA viruses that are spherical, have protrusions, and are crown-like. They are collectively referred to as coronaviruses. The virus has a diameter of 75 to 160 nanometers, and the virus genome is a continuous linear single-stranded RNA, and the molecular weight is usually (5.5 to 6.1)×10₆. The coronavirus genome encodes a spike protein (S), an envelope protein, a membrane protein, and a nucleoprotein in that order.

A coronavirus contains four structural proteins, including spike (S), envelope (E), membrane (M), and nucleocapsid (N) proteins. The S protein functions in viral attachment, fusion, and entry. The S protein mediates viral entry into host cells by first binding to a host receptor through the receptor-binding domain (RBD) in the S1 subunit and then fusing the viral and host membranes through the S2 subunit. See FIG. 1 depicting a sequence alignment of the receptor binding domains from SARS-CoV-2 (SEQ ID NO: 1), SARS-CoV (SEQ ID NO: 2), and MERS-CoV (SEQ ID NO: 3). SARS-CoV and MERS-CoV RBDs recognize different receptors. SARS-CoV recognizes angiotensin-converting enzyme 2 (ACE2) as its receptor, whereas MERS-CoV recognizes dipeptidyl peptidase 4 (DPP4) as its receptor. Similar to SARS-CoV, SARS-CoV-2 also recognizes ACE2 as its host receptor binding to viral S protein. Tai et al. Cellular & Molecular Immunology (2020) 17: 613-620.

### Anti-Coronavirus RBD Antibodies

Anti-coronavirus RBD antibodies may be used in the treatment of coronavirus infections in which a partial or total blockade and/or neutralization of coronavirus activity is desired. In an embodiment, the anti-coronavirus RBD antibodies described herein are used to treat acute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof.

In another aspect, the anti-coronavirus RBD antibodies described herein find utility as reagents for detection and isolation of a coronavirus RBD, such as detection and/or quantification of coronavirus RBD expression in various cells and/or tissues. The anti-coronavirus RBD antibodies described herein can be used in coronavirus RBD binding assays to screen for antagonists of coronavirus RBD which will exhibit similar pharmacological effects.

In an embodiment, antibodies described herein immunospecifically bind a coronavirus RBD polypeptide having the amino acid sequence of SEQ ID NO: 1, 2, 3, 4, or a combination thereof, or a polypeptide comprising a portion (e.g., a fragment) of the amino acid sequence of SEQ ID NO: 1, 2, 3, 4, or a combination thereof. The antibodies described herein including molecules comprising, or alternatively consisting of, antibody fragments or variants thereof that immunospecifically bind to the coronavirus RBD (e.g., a polypeptide comprising, or alternatively consisting of, amino acids 331-524 of the Spike protein of SARS-CoV-2 (SEQ ID NO: 1), amino acid residues 318-510 of the Spike protein of SARS-CoV (SEQ ID NO: 2), amino acid residues 377-588 of the MERS-CoV spike protein (SEQ ID NO: 3). A sequence alignment is provided in FIG. 1. The antibodies and antigen binding fragments described herein preferably bind to an epitope consisting of 6 to 12 residues within the receptor binding domain of amino acids 331-524 of the Spike protein of SARS-CoV-2, amino acid residues 318-510 of the Spike protein of SARS-CoV, amino acid residues 377-588 of the MERS-CoV spike protein.

The antibodies and antigen binding fragments described herein preferably bind to an epitope consisting of 6 to 12 residues within the receptor binding domain of amino acids 319-541 of the SARS CoV-2 spike receptor-binding domain (SEQ ID NO: 4):

RVQPTESIVRFPNITNLCPFGEVFNATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLS
FELLHAPATVCGPKKSTNLVKNKCVNF

Moreover, polypeptide fragments that may be bound by antibodies of the present invention, can be at least 10, 20, 30, 40, 50, 60, 70, 80, 90, 100, 110, 120, 130, 140, 150, 175 or 200 amino acids in length. In this context, “about” means the particularly recited ranges and ranges larger or smaller by several, a few, 5, 4, 3, 2 or 10 amino acid residues at either or both the amino-and carboxy-termini.

Additional embodiments described herein encompass antibodies that bind a coronavirus RBD polypeptide fragments comprising, or alternatively consisting of, functional regions of polypeptides described herein, such as the Gamier-Robson alpha-regions, beta-regions, tum-regions, and coil-regions, Chou-Fasman alpha-regions, beta-regions, and coil-regions, Kyte-Doolittle hydrophilic regions and hydrophobic regions, Eisenberg alpha- and beta-amphipathic regions, Karplus-Schulz flexible regions, Emini surface-forming regions and Jameson-Wolf regions of high antigenic index. In a preferred embodiment, the polypeptide fragments bound by the antibodies described herein are antigenic (e.g., containing four or more contiguous amino acids having an antigenic index of greater than or equal to 1.5, as identified using the default parameters of the Jameson-Wolf program) of a complete (e.g., full-length) coronavirus RBD polypeptide (e.g., SEQ ID NO: 1, 2, 3, 4).

In many embodiments, the antibodies described herein bind a polypeptide comprising, or alternatively consisting of, an epitope-bearing portion of a polypeptide described herein. The epitope of this polypeptide portion may be an immunogenic or antigenic epitope of a polypeptide described herein. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al. Proc. Natl. Acad. Sci. USA 81:3998- 4002 (1983).

As to the selection of polypeptides bearing an antigenic epitope (e.g.., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Leamer, R. A. (1983) “Antibodies that react with predetermined sites on proteins”, Science, 219:660-666. Peptides capable of eliciting protein-reactive sera are frequently represented in the primary sequence of a protein, can be characterized by a set of simple chemical rules, and are confined neither to immunodominant regions of intact proteins (e.g., immunogenic epitopes) nor to the amino or carboxyl terminals. Antigenic epitope-bearing peptides and polypeptides described herein are therefore useful to raise antibodies, including monoclonal antibodies, that bind specifically to a polypeptide described herein. See, for instance, Wilson et al. Cell 37:767-778 (1984) at 777.

In an embodiment, the antibodies described herein bind antigenic epitope-bearing peptides and polypeptides of a coronavirus RBD and preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids contained within the amino acid sequence of a coronavirus RBD polypeptide. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additional non-exclusive preferred antigenic epitopes include the antigenic epitopes disclosed herein, as well as portions thereof.

### Anti-Coronavirus RBD Antibody Epitopes

The present invention encompasses antibodies that bind polypeptides comprising, or alternatively consisting of, an epitope contained within the polypeptide having an amino acid sequence of SEQ ID NO: 1, 2, 3, 4, or a combination thereof.

The antibodies and antigen binding fragments described herein preferably bind to an epitope consisting of 6 to 12 residues within the receptor binding domain of amino acids 331-524 of the Spike protein of SARS-CoV-2 (SEQ ID NO: 1), amino acid residues 318-510 of the Spike protein of SARS-CoV (SEQ ID NO: 2), amino acid residues 377-588 of the MERS-CoV spike protein (SEQ ID NO: 3), and/or the receptor binding domain of amino acids 319-541 of the Spike protein of SARS-CoV-2 (SEQ ID ON: 4).

The term “epitopes,” as used herein, refers broadly to portions of a polypeptide having antigenic or immunogenic activity in an animal, preferably a mammal, and most preferably in a human. In a preferred embodiment, the present invention encompasses antibodies that bind a polypeptide comprising an epitope. An “immunogenic epitope,” as used herein, is defined as a portion of a protein that elicits an antibody response in an animal, as determined by any method known in the art, for example, by the methods for generating antibodies described infra. (See, for example, Geysen et al. Proc. Natl. Acad. Sci. USA 81: 3998- 4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic. Antigenic epitopes are useful, for example, to raise antibodies, including monoclonal antibodies, that specifically bind the epitope. Preferred antigenic epitopes include, but are not limited to, the antigenic epitopes disclosed herein, as well as any combination of two, three, four, five or more of these antigenic epitopes. Antigenic epitopes can be used as the target molecules in immunoassays. (See, for instance, Wilson et al. Cell 37:767-778 (1984); Sutcliffe et al. Science 219:660-666 (1983)).

Similarly, immunogenic epitopes can be used, for example, to induce antibodies according to methods well known in the art. (See, for instance, Sutcliffe et al. supra; Wilson et al. supra; Chow et al. Proc. Natl. Acad. Sci. USA 82:910-914; and Bittle et al. J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes of a coronavirus RBD may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Coronavirus RBD polypeptide fragments which function as epitopes may be produced by any conventional means. (See, e.g., Houghten Proc. Natl. Acad. Sci. USA 82: 5131-5135 (1985) and U.S. Pat. No. 4,631,211).

Epitope-bearing coronavirus RBD polypeptides (SEQ ID NO: 1-4) may be used to induce antibodies according to methods well known in the art including, but not limited to, in vivo immunization, in vitro immunization, and phage display methods. See, e.g., Sutcliffe et al. supra; Wilson et al. supra, and Bittle et al. J. Gen. Virol. 66: 2347-2354 (1985). If in vivo immunization is used, animals may be immunized with free peptide; however, antipeptide antibody titer may be boosted by coupling the peptide to a macromolecular carrier, such as keyhole limpet hemocyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 micrograms of peptide or carrier protein and Freund's adjuvant or any other adjuvant known for stimulating an immune response. Several booster injections may be needed, for instance, at intervals of about two weeks, to provide a useful titer of anti-peptide antibody that can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

### Anti-coronavirus RBD Antibody Fusion Proteins

The antibodies of the present invention may bind polypeptides comprising an immunogenic or antigenic epitope fused to other polypeptide sequences. For example, the coronavirus RBD polypeptides may be fused with the constant domain of immunoglobulins (IgA, IgE, IgG, IgM), or portions thereof (CH1, CH2, CH3, or any combination thereof and portions thereof), or albumin (including but not limited to recombinant human albumin or fragments or variants thereof (See, e.g., U.S. Pat. No. 5,876,969, EP Patent No. 0 413 622, and U.S. Pat. No. 5,766,883, herein incorporated by reference in their entirety), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al. Nature 331: 84-86 (1988). Enhanced delivery of an antigen across the epithelial barrier to the immune system has been demonstrated for antigens (e.g., insulin) conjugated to an FcRn binding partner such as IgG or Fe fragments (See, e.g., PCT Publications WO 96/22024 and WO 99/04813). IgG fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al. J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al. 1991, Proc. Natl. Acad. Sci. USA 88:8972- 897): In this system, the gene of interest is subcloned into a vaccinia recombination plasmid such that the open reading frame of the gene is translationally fused to an amino-terminal tag consisting of six histidine residues. The tag serves as a matrix-binding domain for the fusion protein. Extracts from cells infected with the recombinant vaccinia virus are loaded onto Ni nitriloacetic acid-agarose column and histidine-tagged proteins can be selectively eluted with imidazole-containing buffers.

In another embodiment, the antibodies of the present invention bind a coronavirus RBD polypeptide and/or the epitope-bearing fragments thereof that are fused with a heterologous antigen (e.g., polypeptide, carbohydrate, phospholipid, or nucleic acid). In specific embodiments, the heterologous antigen is an immunogen.

### Anti-Coronavirus RBD Antibody Specificity

The binding specificity of antibodies described herein to a coronavirus RBD polypeptide (SEQ ID NOs: 1-4), or fragments or variants thereof can be determined by any suitable means. Examples of suitable assays to measure binding specificity include, but are not limited to, immunoprecipitation or in vitro binding assays, such as radioimmunoassay (RIA) or enzyme-linked immunoadsorbent assay (ELISA). Other means, such as, surface plasmon resonance may also be used.

The binding affinity of antibodies can, for example, be determined by the Scatchard analysis described by Frankel et al. Mol. Immunol. 16:101-106, 1979. In another embodiment, binding affinity is measured by an antigen/antibody dissociation rate. In another embodiment, a high binding affinity is measured by a competition radioimmunoassay. In another embodiment, binding affinity is measured by ELISA. In another embodiment, antibody affinity is measured by flow cytometry.

An antibody that “specifically binds” or “immunospecifically binds” an antigen (such as coronavirus RBD or fragments or variants thereof) is an antibody that binds the antigen with high affinity and does not significantly bind other unrelated antigens.

In an embodiment, the antibodies described herein bind a coronavirus RBD polypeptide or fragment thereof (such as soluble and/or cell-surface coronavirus RBD) with a dissociation constant (K_{d}) of about 1 nM or less. In an embodiment, the antibodies bind a coronavirus RBD polypeptide or fragment thereof with a binding affinity of about 1 nM, about 0.9 nM, about 0.8 nM, about 0.7 nM, about 0.6 nM, about 0.5 nM, about 0.4 nM, about 0.3 nM, about 0.2 nM, about 0.15 nM, about nM, about 0.05 nM, about 0.04 nM, about 0.03 nM, about 0.02 nM or about 0.01 nM.

Antibodies that bind polypeptides comprising, or alternatively consisting of, a polypeptide having an amino acid sequence least 80%, 85%, 90% identical and more preferably at least 95%, 96%, 97%, 98%, 99% or 100% identical to a coronavirus RBD polypeptide having the amino acid sequence at positions within SEQ ID NO: 1, 2, 3, 4, or a combination thereof are described herein.

Additional embodiments described herein are directed to antibodies that bind polypeptides comprising, or alternatively consisting of, a polypeptide having an amino acid sequence of about 90% to 99% sequence identity to a coronavirus RBD polypeptide having the amino acid sequence of SEQ ID NO: 1, 2, 3, 4, or a combination thereof. The anti-coronavirus-RBD antibodies may selectively bind to a polypeptide having at least about 91%, 92%, 93%, 94%, 95%, 96%, 97%, 98%, 99%, or 100% sequence identity to a coronavirus RBD polypeptide having the amino acid sequence of SEQ ID NO: 1, 2, 3, 4, or a combination thereof.

Antibodies of the present invention may bind fragments, derivatives or analogues of the polypeptide of SEQ ID NO: 1, 2, 3, 4, or a combination thereof, such as (i) polypeptides in which one or more of the amino acid residues are substituted with a conserved or non-conserved amino acid residue (preferably a conserved amino acid residue) and such substituted amino acid residue may or may not be one encoded by the genetic code, or (ii) polypeptides in which one or more of the amino acid residues includes a substituent group, or (iii) polypeptides in which the polypeptide is fused with another compound, such as a compound to increase the half-life of the polypeptide (for example, polyethylene glycol), or (iv) polypeptides in which the additional amino acids are fused to the polypeptide, such as an IgG Fc fusion region peptide or leader or secretory sequence or a sequence which is employed for purification of the polypeptide or a proprotein sequence.

Amino acids in the coronavirus RBD polypeptides that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for functional activity, such ligand binding. Accordingly, antibodies of the present invention may bind amino acids in the coronavirus RBD polypeptides that are essential for function. In an embodiment, antibodies of the present invention bind amino acids in the coronavirus RBD polypeptides that are essential for coronavirus infection. In an embodiment, antibodies of the present invention bind amino acids in the coronavirus RBD polypeptides that inhibit or reduce coronavirus, e.g., SARS-CoV-2, infection. Sites that are critical for ligand-receptor binding can also be determined by structural analysis such as crystallization, nuclear magnetic resonance or photoaffinity labelling (Smith et al. J. Mal. Biol. 224:899-904 (1992) and de Vos et al. Science 255:306-312 (1992)).

### Anti-Coronavirus RBD Antibody Activity

The anti-coronavirus RBD antibodies described herein inhibit the activity of one or more coronaviruses, preferably SARS-CoV-2. Any convenient viral infectivity inhibition assay is suitable for use herein. Such assays are well known in the art. Generally, cells are seeded in attached cell culture plates, grown for 1 day, and then incubated for an additional day in the presence of a predetermined number of units of a selected coronaviruses plus various concentrations of the candidate anti-coronavirus RBD antibody. The candidate anti-coronavirus RBD antibody that inhibits the activity of a coronavirus will inhibit more antiviral activity than the baseline level of antiviral activity inhibition measured in the presence of an equivalent concentration of control antibody.

Optionally, the candidate anti-coronavirus RBD antibody that inhibits the activity of a coronavirus, e.g., SARS-CoV-2, will inhibit at least and/or about 30%, or at least and/or about 40%, or at least and/or about 50%, or at least and/or about 60%, or at least and/or about 70%, or at least and/or about 80%, or at least and/or about 90%, or at least and/or about 95%, or at least and/or about 96%, or at least and/or about 97%, or at least and/or about 98%, or at least and/or about 99%, or about 100% of the activity of the coronavirus in the viral activity assay as compared to baseline activity measured in the presence of an equivalent concentration of control antibody. The candidate anti-coronavirus RBD antibody that does not inhibit the activity of a selected coronavirus will exhibit similar or approximately the same level of antiviral activity inhibition as a control antibody.

If an anti-coronavirus RBD monoclonal antibody that binds to a particular a coronavirus RBD determinant(s) is desired, the candidate antibody can be screened for the presence or absence of differential affinity to wild type coronavirus RBD and to mutant coronavirus RBD that contains Ala substitution(s) at the determinant(s) of interest as described above. In one aspect, the candidate antibody can be tested for binding to wild type coronavirus RBD and mutant coronavirus RBD in an immunoprecipitation or immunoadsorption assay. For example, a capture ELISA can be used wherein plates are coated with a given concentration of wild type coronavirus RBD or an equal concentration of mutant coronavirus RBD, the coated plates are contacted with equal concentrations of the candidate antibody, and the bound antibody is detected enzymatically, e.g., contacting the bound antibody with HRP-conjugated anti-Ig antibody and developing the HRP color reaction. The candidate antibody that binds to the particular coronavirus RBD determinant(s) of interest will exhibit binding activity with wild type coronavirus RBD that is greater than the candidate antibody's binding activity with the corresponding Ala-substituted coronavirus RBD mutant (e.g., a binding level with wild type coronavirus RBD that is above the background binding level with mutant coronavirus RBD).

Optionally, the candidate antibody that binds to the particular coronavirus RBD determinant(s) of interest will exhibit binding activity with the corresponding Ala-substituted coronavirus RBD mutant that is less than about 50%, or less than about 30%, or less than about 20%, or less than about 10%, or less than about 7%, or less than about 6%, or less than about 5%, or less than about 4%, or less than about 3%, or less than about 2%, or less than about 1%, or about 0% of the antibody's binding activity with wild type coronavirus RBD, e.g., as determined by dividing the HRP color reaction optical density observed for capture ELISA with coronavirus RBD mutant adsorbent by the HRP color reaction optical density observed for capture ELISA with wild type coronavirus RBD adsorbent.

The anti-coronavirus RBD antibodies described herein may possess combinations of the coronavirus activity inhibition and the coronavirus RBD determinant binding properties described herein. Anti-coronavirus RBD antibodies corresponding to these embodiments can be obtained by using combinations of coronavirus competitive binding and/or activity inhibition assays described herein for selection of antibodies with coronavirus inhibiting properties and the immunoprecipitation or immunoadsorption screening procedures described herein for selection of antibodies with unique coronavirus RBD determinant binding properties.

The anti-coronavirus RBD antibodies described herein may neutralize one or more coronavirus. For example, the anti-coronavirus RBD antibodies described herein may neutralize SARS-CoV-1, MERS-CoV, SARS-CoV-2 (COVID-19), HCoV-0C43, HCoV-HKU1, HCoV-NL63, HCoV-229E or a combination thereof. The antibodies described herein may neutralize coronaviruses infecting any animal, preferably from a mammal. Most preferably, the antibodies described herein neutralize coronaviruses infecting a human.

### Anti-Coronavirus RBD Antibodies

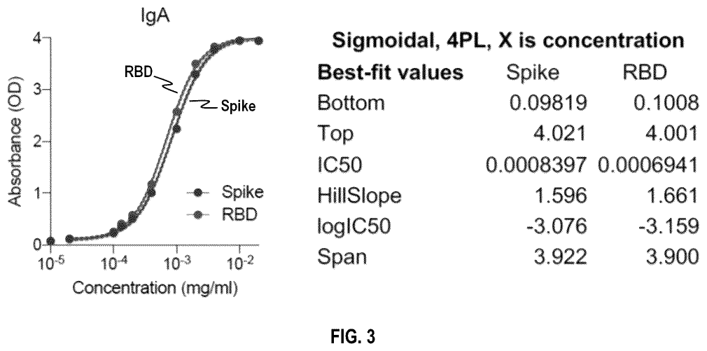

Provided herein are antibodies that immunospecifically bind to a coronavirus RBD with high affinity and neutralize coronaviruses, preferably SARS-CoV-1, MERS-CoV, SARS-CoV-2 (COVID-19), HCoV-0C43, HCoV-HKU1, HCoV-NL63, HCoV-229E or a combination thereof. These antibodies have been demonstrated, for example, to strongly bind to both an isolated receptor binding domain (RBD) and a full spike ectodomain trimer from SARS-CoV-2. See FIG. 3. The antibodies described herein may comprise a variable region of the light chain (SEQ ID NO: 23) comprising (in order) light-chain frame work 1 (SEQ ID NO: 9), light-chain CDR1 (SEQ ID NO: 10), light-chain frame work 2 (SEQ ID NO: 11), light-chain CDR2 (SEQ ID NO: 12), light-chain frame work 3 (SEQ ID NO: 13), light-chain CDR3 (SEQ ID NO: 14), and light-chain frame work 4 (SEQ ID NO: 15) and a variable region of the heavy chain (SEQ ID NO: 24) comprising (in order) heavy-chain frame work 1 (SEQ ID NO: 16), heavy-chain CDR1 (SEQ ID NO: 17), heavy-chain frame work 2 (SEQ ID NO: 18), heavy-chain CDR2 (SEQ ID NO: 19), heavy-chain frame work 3 (SEQ ID NO: 20), heavy-chain CDR3 (SEQ ID NO: 21), and heavy-chain frame work 4 (SEQ ID NO: 22).

The antibody or fragment thereof described herein comprises a light chain variable region of the amino acid sequence of SEQ ID NO: 23. The antibody or fragment thereof described herein comprises a light chain variable region comprising light chain frame work sequences comprising the amino acid sequences of SEQ ID NO: 9, 11, 13, and/or 15. The antibody or fragment thereof described herein comprises a light chain variable region comprising CDRs sequences comprising the amino acid sequences of SEQ ID NO: 10, 12, and/or 14.

The antibody or fragment thereof described herein comprises a light chain variable region of the amino acid sequence sharing about 80% sequence homology with the amino acid sequence of SEQ ID NO: 23. The antibody or fragment thereof described herein comprises a light chain variable region comprising light chain frame work sequences comprising the amino acid sequences sharing about 80% sequence homology with the amino acid sequences of SEQ ID NO: 9, 11, 13, and/or 15. The antibody or fragment thereof described herein comprises a light chain variable region comprising CDRs sequences comprising amino acid sequences sharing about 80% sequence homology with the amino acid sequences of SEQ ID NO: 10, 12, and/or 14. The sequence homology may be at least about 85%, 90%, 91%, 92%, 93%, 94%, 95%, 96%, 97%, 98%, 99%, or 100% sequence homology.

The antibody or fragment thereof described herein comprises a heavy chain variable region of the amino acid sequence of SEQ ID NO: 24. The antibody or fragment thereof described herein comprises a heavy chain variable region comprising heavy chain frame work sequences comprising the amino acid sequences of SEQ ID NO: 16, 18, 20, and/or 22. The antibody or fragment thereof described herein comprises a heavy chain variable region comprising CDRs sequences comprising the amino acid sequences of SEQ ID NO: 17, 19, and/or 21.

The antibody or fragment thereof described herein comprises a heavy chain variable region comprising an amino acid sequence sharing about 80% sequence homology with the amino acid sequence of SEQ ID NO: 24. The antibody or fragment thereof described herein comprises a heavy chain variable region comprising heavy chain frame work sequences comprising amino acid sequences sharing about 80% sequence homology with the amino acid sequences of SEQ ID NO: 16, 18, 20, and/or 22. The antibody or fragment thereof described herein comprises a heavy chain variable region comprising CDRs sequences comprising amino acid sequences sharing about 80% sequence homology with the amino acid sequences of SEQ ID NO: 17, 19, and/or 21. The sequence homology may be at least about 85%, 90%, 91%, 92%, 93%, 94%, 95%, 96%, 97%, 98%, 99%, or 100% sequence homology.

The amino acid sequences of heavy chain and light chain variable regions of the anti-coronavirus RBD antibodies are shown below.

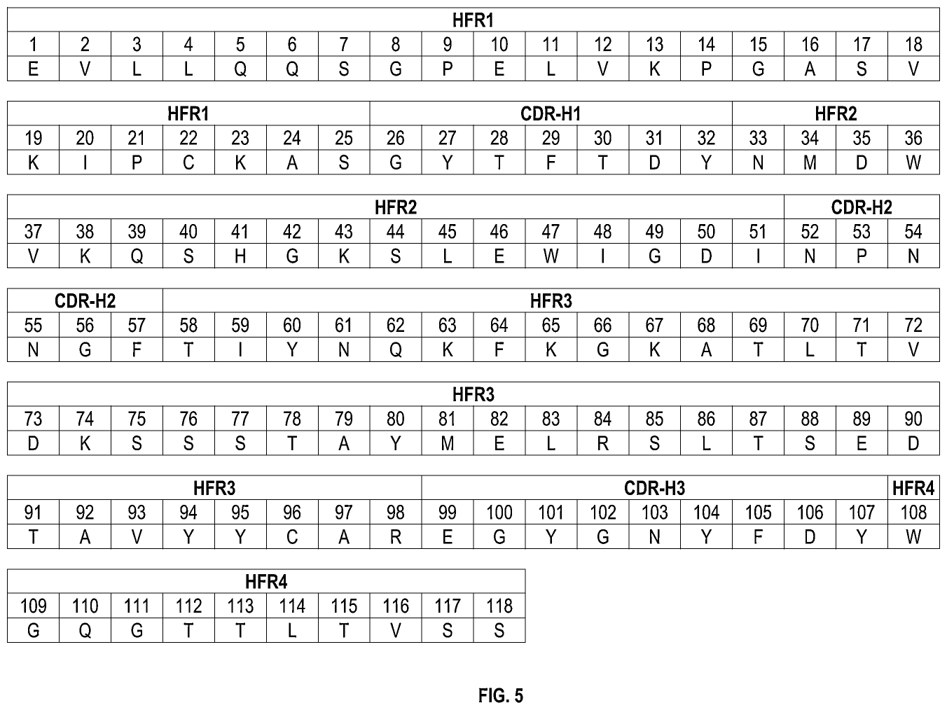

Human IgA heavy chain variable region [SEQ ID NO: 24; see also FIG. 5]

```
EVLLQQSGPELVKPGASVKIPCKASGYTFTDYNMDWVKQSHGKSLEWIGDINPNNGFTIYN
      framework 1 CDR1 framework 2 CDR2
QKFKGKATLTVDKSSSTAYMELRSLTSEDTAVYYCAREGYGNYFDYWGQGTTLTVSS
                framework 3 CDR3 framework 4
```

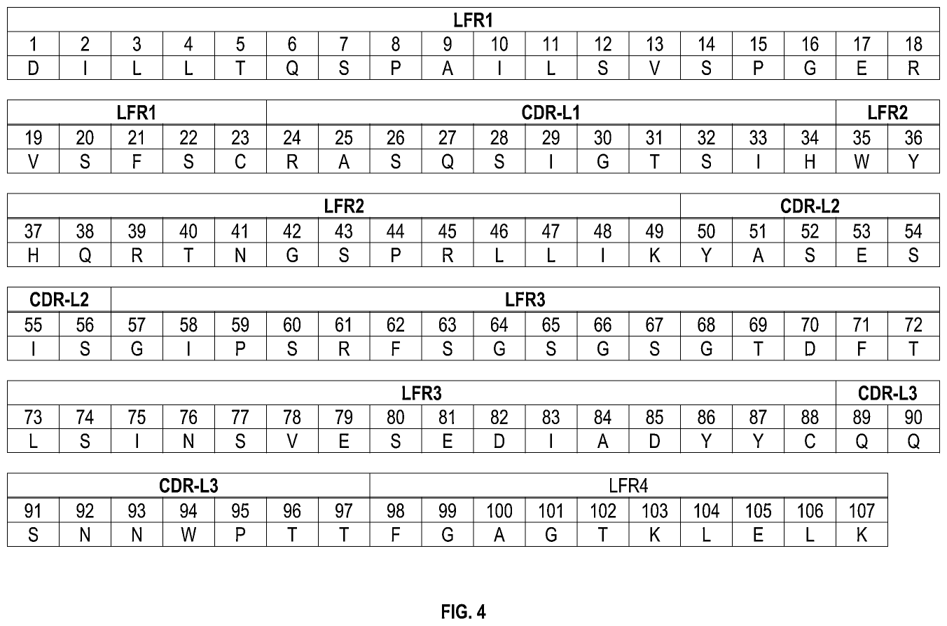

Human IgA light chain variable region [SEQ ID NO: 23; see also FIG. 4]

```
DILLTQSPAILSVSPGERVSFSCRASQSIGTSIHWYHQRTNGSPRLLIKYASESISGIPSRFSGSG
          framework 1 CDR1 framework 2 CDR2
SGTDFTLSINSVESEDIADYYCQQSNNWPTTFGAGTKLELK
          framework 3 CDR3 framework 4
```

CDR annotation with Chothia scheme.

The amino acid sequences of heavy chain constant region and light chain constant region of the anti-coronavirus RBD antibodies are shown below.

Human IgA heavy chain constant region (IGHA1) [SEQ ID NO: 5]

ASPTSPKVFP LSLCSTQPDG NVVIACLVQG FFPQEPLSVT
WSESGQGVTA RNFPPSQDAS GDLYTTSSQL TLPATQCLAG
KSVTCHVKHY TNPSQDVTVP CPVPSTPPTP SPSTPPTPSP
SCCHPRLSLH RPALEDLLLG SEANLTCTLT GLRDASGVTF
TWTPSSGKSA VQGPPERDLC GCYSVSSVLP GCAEPWNHGK
TFTCTAAYPE SKTPLTATLS KSGNTFRPEV HLLPPPSEEL
ALNELVTLTC LARGFSPKDV LVRWLQGSQE LPREKYLTWA
SRQEPSQGTT TFAVTSILRV AAEDWKKGDT FSCMVGHEAL
PLAFTQKTID RLAGKPTHVN VSVVMAEVDG TCY

Light chain constant region Amino Acid Sequence (SEQ ID NO: 6)

TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC

The antibody or fragment thereof described herein comprises a heavy chain constant region of the amino acid sequence of SEQ ID NO: 5.

The antibody or fragment thereof described herein comprises a light chain constant region of the amino acid sequence of SEQ ID NO: 6.

TABLE 1
CDR sequences for anti-coronavirus RBD antibody

| | Amino Acid Sequence |
|---|---|
| VHCDR 1 | (SEQ ID NO: 17) |
| VHCDR 2 | (SEQ ID NO: 19) |
| VHCDR 3 | (SEQ ID NO: 21) |
| VLCDR 1 | (SEQ ID NO: 10) |
| VLCDR 2 | (SEQ ID NO: 12) |
| VLCDR 3 | (SEQ ID NO: 14) |

CDR annotation with Chothia scheme.

The antibody or antigen-binding fragment thereof described herein comprises a VH domain of SEQ ID NO: 24.

The antibody or antigen-binding fragment thereof comprises one, two, or all three CDRS of the VH domain of SEQ ID NO: 8. For example, the antibody or fragment thereof may comprise a CDR1 of SEQ ID NO: 17, a CDR2 of SEQ ID NO: 19, a CDR3 of SEQ ID NO: 21, or any combination thereof. The antibody or antigen-binding fragment thereof may comprise all three heavy chain variable region CDR sequences, a CDR1 of SEQ ID NO: 17, a CDR2 of SEQ ID NO: 19, and a CDR3 of SEQ ID NO: 21.

The antibody or antigen-binding fragment thereof described herein comprises a VL domain of SEQ ID NO: 23.

The antibody or antigen-binding fragment thereof comprises one, two, or all three CDRs of the VL domain of SEQ ID NO: 23. For example, the antibody or fragment thereof may comprise a CDR1 of SEQ ID NO: 10, a CDR2 of SEQ ID NO: 12, a CDR3 of SEQ ID NO: 14, or any combination thereof. The antibody or antigen-binding fragment thereof may comprise all three light chain variable region CDR sequences, a CDR1 of SEQ ID NO: 10, a CDR2 of SEQ ID NO: 12, and a CDR3 of SEQ ID NO: 14.

The antibody or antigen-binding fragment thereof comprises a VH domain of SEQ ID NO: 24 and a VL domain of SEQ ID NO: 23.

The antibody or antigen-binding fragment thereof comprises one, two, or all three CDRs of the VH domain of SEQ ID NO: 24 and one, two, or all three CDRs of the VL domain of SEQ ID NO: 23. For example, the antibody or fragment thereof may comprise a light-chain CDR1 of SEQ ID NO: 10, a light-chain CDR2 of SEQ ID NO: 12, a light-chain CDR3 of SEQ ID NO: 14, or any combination thereof and a heavy-chain CDR1 of SEQ ID NO: 17, a heavy-chain CDR2 of SEQ ID NO: 19, a heavy-chain CDR3 of SEQ ID NO: 21, or any combination thereof.

In an embodiment, the antibody or fragment thereof comprises all three CDRs of the VH domain of SEQ ID NO: 24 and all three CDRs of the VL domain of SEQ ID NO: 23. For example, the antibody or fragment thereof comprises a VHCDR1 of SEQ ID NO: 17, a VHCDR2 of SEQ ID NO: 19, a VHCDR3 of SEQ ID NO: 21, a VLCDR1 of SEQ ID NO: 10, a VLCDR2 of SEQ ID NO: 12, and a VLCDR3 of SEQ ID NO: 14.

### Anti-Coronavirus RBD Antibody Variants

The antibodies described herein may be from any animal origin, including birds and mammals. Preferably, the antibodies are human, murine (e.g., mouse and rat), donkey, sheep, rabbit, goat, guinea pig, camel, horse, or chicken. In an embodiment, the antibodies are human antibodies. As used herein, “human” antibodies include antibodies having the amino acid sequence of a human immunoglobulin and include antibodies isolated from human immunoglobulin libraries and xenomice or other organisms that have been genetically engineered to produce human antibodies. For a detailed discussion of a few of the technologies for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598; and Lenberg and Huszar, Int. Rev. Immunol. 13:65-93 (1995), which are incorporated by reference herein in their entirety. Human antibodies or “humanized” chimeric monoclonal antibodies can be produced using techniques described herein or otherwise known in the art. For example, methods for producing chimeric antibodies are known in the art. See, for review the following references which are hereby incorporated in their entirety: Morrison, Science 229:1202 (1985); Oi et al. BioTechniques 4:214 (1986); Cabilly et al. U.S. Patent No. 4,816,567; Taniguchi et al. EP 171496; Morrison et al. EP 173494; Neuberger et al. WO 8601533; Robinson et al. WO 8702671; Boulianne et al. Nature 312:643 (1984); Neuberger et al. Nature 314:268 (1985).

### Chimeric Anti-Coronavirus RBD Antibodies

A chimeric antibody is a molecule in which different portions of the antibody are derived from different immunoglobulin molecules such as antibodies having a variable region derived from a human antibody and a non-human immunoglobulin constant region. Methods for producing chimeric antibodies are known in the art. See e.g., Morrison, Science 229:1202 (1985); Oi et al. BioTechniques 4:214 (1986); Gillies et al. J. Immunol. Methods 125:191-202 (1989); U.S. Pat. Nos. 5,807,715; 4,816,567; and 4,816,397, which are incorporated herein by reference in their entirety. Chimeric antibodies comprising one or more CDRs from human species and framework regions from a non-human immunoglobulin molecule (e.g., framework regions from a canine or feline immunoglobulin molecule) can be produced using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al. Protein Engineering 7(6):805-814 (1994); Roguska et al. PNAS 91 :969-973 (1994)), and chain shuffling (U.S. Pat. No. 5,565,332). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al. U.S. Pat. No. 5,585,089; Riechmann et al. Nature 332:323 (1988), which are incorporated herein by reference in their entireties.

### Anti-Coronavirus RBD Antibody Mimics

Further, the antibodies described herein can, in turn, be utilized to generate antibodies that “mimic” RBD polypeptides using techniques well known to those skilled in the art. (See, e.g., Greenspan & Bona, FASEB J. 7(5): 437-444 (1993); and Nissinoff, J. Immunol. 147(8):2429-2438 (1991)). For example, antibodies described herein which bind to a coronavirus RBD and competitively inhibit the binding of coronavirus to a cell (as determined by assays well known in the art such as, for example, that disclosed, infra) can be used to generate anti-idiotypes that “mimic” a coronavirus RBD-cell binding to the cell receptor ACE2 and, as a consequence, bind to and neutralize coronaviruses. Such neutralizing anti-idiotypes (including molecules comprising, or alternatively consisting of, antibody fragments or variants, such as Fab fragments of such anti-idiotypes) can be used in therapeutic regimens to neutralize coronaviruses. For example, such anti-idiotypic antibodies can be used to bind coronavirus RBD, and thereby block coronaviruses from binding cells, e.g., ACE2 receptor.

### Monoclonal Anti-Coronavirus RBD Antibodies

The monoclonal antibodies disclosed herein can be of any isotype. The monoclonal antibody can be, for example, IgA, IgD, IgE, IgM, or an IgG antibody, such as IgG1 or an IgG2. The class of an antibody that immunospecifically binds a coronavirus RBD can be switched with another (for example, IgG can be switched to IgM), according to well-known procedures. Class switching can also be used to convert one IgG subclass to another, such as from IgG1 to IgG2. The monoclonal antibodies described herein are IgA, including serum IgA and secretory IgA (sIgA), more preferably secretory IgA (sIgA).

The antibodies of the present invention may be monovalent, bivalent, trivalent or multivalent. For example, monovalent scFvs can be multimerized either chemically or by association with another protein or substance. An scFv that is fused to a hexahistidine tag or a Flag tag can be multimerized using Ni-NTA agarose (Qiagen) or using anti-Flag antibodies (Stratagene, Inc.).

The antibodies of the present invention may be monospecific, bispecific, trispecific or of greater multispecificity. Multispecific antibodies may be specific for different epitopes of a coronavirus RBD polypeptide, or fragment thereof, or may be specific for both a coronavirus RBD polypeptide, or fragment thereof, and a heterologous epitope, such as a heterologous polypeptide or solid support material. See, e.g., PCT publications WO 93/17715; WO 92/08802; WO 91/00360; WO 92/05793; Tutt, et al. J. Immunol. 147:60-69 (1991); U.S. Pat. Nos. 4,474,893; 4,714,681; 4,925,648; 5,573,920; 5,601,819; Kostelny et al. J. Immunol. 148:1547-1553 (1992).

### Anti-Coronavirus RBD Antibody Cross-reactivity

Antibodies of the present invention may also be described or specified in terms of their cross-reactivity. Antibodies that do not bind any other analog, orthologue, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention.

Antibodies of the present invention may cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under hybridization conditions (as described herein).

In an embodiment, the antibodies of the present invention (including molecules comprising, or alternatively consisting of, antibody fragments or variants thereof), immunospecifically bind to a coronavirus RBD and do not cross-react with any other antigens.

### Anti-Coronavirus RBD Variants and Derivatives

The present invention also provides antibodies that comprise, or alternatively consist of, variants (including derivatives) of the VH domains, VH CDRs, VL domains, and VL CDRs described herein, which antibodies immunospecifically bind to a coronavirus RBD. Standard techniques known to those of skill in the art can be used to introduce mutations in the nucleotide sequence encoding a molecule described herein, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH domain (SEQ ID NO: 24), VHCDR1 (SEQ ID NO: 17), VHCDR2 (SEQ ID NO: 19), VHCDR3 (SEQ ID NO: 21), VL domain (SEQ ID NO: 23), VLCDR1 (SEQ ID NO: 10), VLCDR2 (SEQ ID NO: 12), or VLCDR3 (SEQ ID NO: 14). In an embodiment, the variants have conservative amino acid substitutions at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a coronavirus RBD). Following mutagenesis, the encoded protein may routinely be expressed and the functional and/or biological activity of the encoded protein, (e.g., ability to immunospecifically bind a coronavirus RBD) can be determined using techniques described herein or by routinely modifying techniques known in the art.

The antibodies described herein include derivatives (e.g., variants) that are modified, e.g., by the covalent attachment of any type of molecule to the antibody such that covalent attachment does not affect the ability of the antibody to immunospecifically bind to a coronavirus RBD. For example, but not by way of limitation, derivatives described herein include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

### Anti-Coronavirus RBD Antibodies Sequences and Structure

In an embodiment, an antibody described herein (including a molecule comprising, or alternatively consisting of, an antibody fragment or variant thereof), that immunospecifically binds a coronavirus RBD, comprises, or alternatively consists of, an amino acid sequence encoded by a nucleotide sequence that hybridizes to a nucleotide sequence that is complementary to that encoding one of the VH (SEQ ID NO: 24) or VL (SEQ ID NO: 23) domains disclosed herein under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F.M. et al. eds., 1989, Current Protocols in Molecular Biology, Vol. I, Green Publishing Associates, Inc. and John Wiley & Sons, Inc., New York at pages 6.3.1-6.3.6 and 2.10.3). In another embodiment, an antibody described herein that immunospecifically binds to a coronavirus RBD, comprises, or alternatively consists of, an amino acid sequence encoded by a nucleotide sequence that hybridizes to a nucleotide sequence that is complementary to that encoding one of heavy chain (SEQ ID NO: 8), heavy chain constant region (SEQ ID NO: 5), heavy chain variable region (SEQ ID NO: 24), light chain (SEQ ID NO: 7), light chain constant region (SEQ ID NO: 6), light chain variable region (SEQ ID NO: 23), disclosed herein under stringent conditions, e.g., hybridization under conditions as described above, or under other stringent hybridization conditions which are known to those of skill in the art. Nucleic acid molecules encoding these antibodies are also encompassed by the invention.

The invention also encompasses antibodies (including molecules comprising, or alternatively consisting of, antibody fragments or variants thereof) that have one or more of the same biological characteristics as one or more of the antibodies described herein. By “biological characteristics” is meant, the in vitro or in vivo activities or properties of the antibodies, such as, for example, the ability to bind to a coronavirus RBD and/or an antigenic and/or epitope region of a coronavirus RBD), the ability to substantially block coronavirus binding to its target, or the ability to block coronavirus biological activity. Optionally, the antibodies described herein will bind to the same epitope as at least one of the antibodies specifically referred to herein. Such epitope binding can be routinely determined using assays known in the art.

By an antibody that “neutralizes coronavirus” is meant an antibody that diminishes or abolishes the ability of a coronavirus to bind to a coronavirus RBD. In one embodiment, an antibody that neutralizes Coronavirus, e.g., SARS-CoV-2, comprises, or alternatively consists of, a polypeptide comprising the amino acid sequence of a heavy chain consisting of the amino acid sequence of SEQ ID NO: 8, or a fragment thereof and a light chain consisting of the amino acid sequence of SEQ ID NO: 7, or a fragment thereof. In an embodiment, an antibody that neutralizes Coronavirus, e.g., SARS-CoV-2, comprises, or alternatively consists of, a polypeptide comprising the amino acid sequence of a heavy chain constant domain consisting of the amino acid sequence of SEQ ID NO: 5, or a fragment thereof, a light chain constant domain consisting of the amino acid sequence of SEQ ID NO: 6, or a fragment thereof, a light chain variable domain consisting of the amino acid sequence of SEQ ID NO: 23, or a fragment thereof, and a heavy chain variable domain consisting of the amino acid sequence of SEQ ID NO: 24, or a fragment thereof. Nucleic acid molecules encoding these antibodies are also encompassed by the invention.

The antibodies described herein also include but are not limited to antibodies that competitively inhibit binding of an antibody comprising a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide. In preferred embodiments, the invention provides antibodies which reduce the binding of an antibody comprising a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by between 1% and 10% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 10% and up to 20% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 20% and up to 30% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 30% and up to 40% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 40% and up to 50% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 50% and up to 60% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 60% and up to 70% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 70% and up to 80% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 80% and up to 90% in a competitive inhibition assay.

In an embodiment, antibodies which reduce the binding of an antibody may comprise a fragment (e.g., VH domain (SEQ ID NO: 24), VL domain (SEQ ID NO: 23), VHCDR1 (SEQ ID NO: 17), VHCDR2 (SEQ ID NO: 19), VHCDR3 (SEQ ID NO: 21), VLCDR1 (SEQ ID NO: 10), VLCDR2 (SEQ ID NO: 12), or VLCDR3 (SEQ ID NO: 14)) described herein or variant thereof to a coronavirus RBD polypeptide by at least 90% and up to 100% in a competitive inhibition assay.

The present invention also provides for mixtures of antibodies (including scFvs and other molecules comprising, or alternatively consisting of, antibody fragments or variants thereof) that immunospecifically bind to a coronavirus RBD, wherein the mixture has at least one, two, three, four, five or more different antibodies described herein. In particular, the invention provides for mixtures of different antibodies that immunospecifically bind to the coronavirus RBD. In an embodiment, the invention provides mixtures of at least 2, preferably at least 4, at least 6, at least 8, at least 10, at least 12, at least 15, at least 20, or at least 25 different antibodies that immunospecifically bind to a coronavirus RBD, wherein at least 1, at least 2, at least 4, at least 6, or at least 10, antibodies of the mixture is an antibody described herein. In an embodiment, each antibody of the mixture is an antibody described herein.

### Panels of Antibodies

The present invention also provides for panels of antibodies (including scFvs and other molecules comprising, or alternatively consisting of, antibody fragments or variants thereof) that immunospecifically bind to a coronavirus RBD, wherein the panel has at least one, two, three, four, five or more different antibodies described herein. In particular, the invention provides for panels of different antibodies that immunospecifically bind to the coronavirus RBD. In an embodiment, the invention provides for panels of antibodies that have different affinities for a coronavirus RBD, different specificities for a coronavirus RBD, or different dissociation rates. The invention provides panels of at least 10, preferably at least 25, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, or at least 1000, antibodies. Panels of antibodies can be used, for example, in 96 well plates for assays such as ELISAs.

### Compositions

The present invention further provides for compositions comprising, one or more antibodies (including scFvs and other molecules comprising, or alternatively consisting of antibody fragments or variants described herein). In one embodiment, a composition of the present invention comprises, one, two, three, four, five, or more antibodies that comprise or alternatively consist of, a polypeptide having an amino acid sequence of any one or more of the heavy chain constant regions comprising the amino acid sequence of SEQ ID NO: 5 or a variant thereof. In another embodiment, a composition of the present invention comprises, one, two, three, four, five, or more antibodies that comprise, or alternatively consist of, a polypeptide having an amino acid sequence of any one or more of the light chain constant region of the amino acid sequence of SEQ ID NO: 6 or a variant thereof. In one embodiment, a composition of the present invention comprises, one, two, three, four, five, or more antibodies that comprise or alternatively consist of, a polypeptide having an amino acid sequence of any one or more of the heavy chain variable regions comprising the amino acid sequence of SEQ ID NO: 24 or a variant thereof. In another embodiment, a composition of the present invention comprises, one, two, three, four, five, or more antibodies that comprise, or alternatively consist of, a polypeptide having an amino acid sequence of any one or more of the light chain variable region of the amino acid sequence of SEQ ID NO: 23 or a variant thereof.

A composition described herein may comprise, one, two, three, four, five, or more antibodies that comprise, or alternative consist of, the heavy chain constant regions comprising the amino acid sequence of SEQ ID NO: 5 or a variant thereof and a light chain constant region of the amino acid sequence of SEQ ID NO: 6 or a variant thereof.

A composition described herein may comprise, one, two, three, four, five, or more antibodies that comprise, or alternative consist of, the heavy chain variable regions comprising the amino acid sequence of SEQ ID NO: 24 or a variant thereof and a light chain variable region of the amino acid sequence of SEQ ID NO: 23 or a variant thereof.

As discussed in more detail below, a composition described herein may be used either alone or in combination with other compositions. The antibodies (including scFvs and other molecules comprising, or alternatively consisting of antibody fragments or variants of the present invention) may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Patent No. 5,314,995; and EP 396,387.

The composition described herein may be a pharmaceutical composition. The composition, including pharmaceutical compositions, may comprise an antibody or antigen-binding fragment described herein and an adjuvant, carrier, buffers, antioxidants, wetting agents, lubricating agents, gelling agents, thickening agents, binding agents, disintegrating agents, humectants, preservatives, diluent, stabilizer, filler, excipient, or a combination thereof.

The antibodies described herein may be formulated for administration by inhalation, preferably intranasal administration. The antibodies described herein may be lyophilized, preferably stabilized, for administration by inhalation, preferably intranasal.

Antibodies described herein (including scFvs and other molecules comprising, or alternatively consisting of antibody fragments or variants of the present invention) may be used, for example, but not limited to, to purify and detect a coronavirus RBD, and to target the polypeptides of the present invention to cells expressing a coronavirus RBD, including both in vitro and in vivo diagnostic and therapeutic methods. For example, the antibodies have use in immunoassays for qualitatively and quantitatively measuring levels of a coronavirus RBD in biological samples. See, e.g., Harlow et al. Antibodies: A Laboratory Manual, (Cold Spring Harbor Laboratory Press, 2nd ed. 1988) (incorporated by reference herein in its entirety).

### Nucleic Acids, Vectors, and Host Cells

The present invention also provides for an isolated nucleic acid molecule encoding an antibody described herein (including molecules comprising, or alternatively consisting of, antibody fragments or variants thereof).

Nucleic acid molecules that encode the anti-coronavirus RBD antibodies described herein. The nucleic acids may be present in whole cells, in a cell lysate, or in a partially purified or substantially pure form. A nucleic acid may be isolated by purification away from other cellular components or other contaminants (e.g., other cellular nucleic acids or proteins) by standard techniques, including alkaline/SDS treatment, CsCI banding, column chromatography, agarose gel electrophoresis and others well known in the art. See Ausubel, et al. (2011) Current Protocols in Molecular Biology John Wiley & Sons, Inc. A nucleic acid described herein may be, for example, DNA or RNA and may or may not contain intronic sequences. The nucleic acid may be a cDNA molecule. Nucleic acids described herein may be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma may be obtained by standard PCR amplification or cDNA cloning techniques. For antibodies obtained from an immunoglobulin gene library (e.g., using phage display techniques), nucleic acid encoding the antibody may be recovered from the library. Specifically, degenerate codon substitutions may be achieved by generating, e.g., sequences in which the third position of one or more selected codons is substituted with mixed-base and/or deoxyinosine residues. Batzer, et al. (1991) Nucleic Acid Res. 19: 5081; Ohtsuka, et al. (1985) J. Biol. Chem. 260: 2605-08; Rossolini, et al. (1994) Mol. Cell. Probes 8: 91-98.

### Methods for Producing Antibodies

The antibodies described herein (including scFvs and other molecules comprising, or alternatively consisting of antibody fragments or variants described herein) can be produced by any method known in the art for the synthesis of antibodies, in particular, by chemical synthesis or preferably, by recombinant expression techniques.

Single chain Fvs (scFvs) that immunospecifically bind a coronavirus RBD may be generated using phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues) or synthetic cDNA libraries. The DNA encoding the VH (SEQ ID NO: 24) and VL (SEQ ID NO: 23) domains are joined together by an scFv linker by PCR and cloned into a phagemid vector (e.g., p CANT AB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an antigen of interest (e.g., coronavirus RBD or a fragment or variant thereof) can be selected or identified with antigen, e.g., using labeled antigen or antigen bound or captured to a solid surface or bead. Examples of phage display methods that can be used to make the antibodies of the present invention include, but are not limited to, those disclosed in Brinkman et al. J. Immunol. Methods 182:41-50 (1995); Ames et al. J. Immunol. Methods 184:177-186 (1995); Kettleborough et al. Eur. J. Immunol. 24:952-958 (1994); Persic et al. Gene 187 9-18 (1997); Burton et al. Advances in Immunology 57:191-280(1994); International Patent Application No. PCT/GB91/01134; WO 90/02809; WO 91/10737; WO 92/01047; WO 92/18619; WO 93/11236; WO 95/15982; WO 95/20401; WO 97/13844; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

As described in the above references, after phage selection, the antibody coding regions from the phage can be isolated and used to generate whole antibodies, including human or humanized antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication WO 92/22324; Mullinax et al. BioTechniques 12(6):864-869 (1992); Sawai et al. AJRI 34:26-34 (1995); and Better et al. Science 240:1041-1043 (1988) (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lambda constant regions. Preferably, the vectors for expressing the VH or VL domains comprise a promoter suitable to direct expression of the heavy and light chains in the chosen expression system, a secretion signal, a cloning site for the immunoglobulin variable domain, immunoglobulin constant domains, and a selection marker such as neomycin. The VH and VL domains may also be cloned into one vector expressing the necessary constant regions. The heavy chain conversion vectors and light chain conversion vectors are then co-transfected into cell lines to generate stable or transient cell lines that express full-length antibodies, e.g., lgG, using techniques known to those of skill in the art.

Once an antibody molecule described herein (including molecules comprising, or alternatively consisting of, antibody fragments or variants thereof) has been chemically synthesized or recombinantly expressed, it may be purified by any method known in the art for purification of an immunoglobulin molecule, or more generally, a protein molecule, such as, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention may be fused to heterologous polypeptide sequences described herein or otherwise known in the art, to facilitate purification.

The present invention also provides methods for recombinantly producing the anti-coronavirus RBD antibodies described herein. Methods of producing the antibodies described herein are well known to those of ordinary skill in the art. The anti-coronavirus RBD antibodies described herein may also be produced by constructing, using conventional techniques well known to those of ordinary skill in the art, an expression vector containing an operon and a DNA sequence encoding the anti-coronavirus RBD antibodies described herein. Furthermore, the invention relates to vectors, especially plasmids, cosmids, viruses, bacteriophages and other vectors common in genetic engineering, which contain the above-mentioned nucleic acid molecules described herein. The nucleic acid molecules contained in the vectors may be linked to regulatory elements that ensure the transcription in prokaryotic and eukaryotic cells.

Vectors contain elements that facilitate manipulation for the expression of a foreign protein within the target host cell. Conveniently, manipulation of sequences and production of DNA for transformation is first performed in a bacterial host (e.g., E. coli) and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that confer resistance to antibiotics or other toxins, complement auxotrophic deficiencies, or supply critical nutrients not available from complex media. Exemplary vectors and methods for transformation of yeast are described in the art. See, e.g., Burke, et al. (2000) Methods in Yeast Genetics Cold Spring Harbor Laboratory Press.

The polynucleotide coding for the anti-coronavirus RBD antibodies may be operably linked to transcriptional and translational regulatory sequences that provide for expression of the polypeptide in yeast cells. These vector components may include, but are not limited to, one or more of the following: an enhancer element, a promoter, and a transcription termination sequence. Sequences for the secretion of the polypeptide may also be included (e.g., a signal sequence).

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” refers broadly to contiguous linked DNA sequences, and, in the case of a secretory leader, contiguous and in reading frame. However, enhancers do not have to be contiguous.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (e.g., that increase levels of transcription in response to absence of a repressor). Inducible promoters may initiate increased levels of transcription from DNA under their control in response to some change in culture conditions (e.g., the presence or absence of a nutrient or a change in temperature.)

The expression vectors are transfected into a host cell by convention techniques well known to those of ordinary skill in the art to produce a transfected host cell, said transfected host cell cultured by conventional techniques well known to those of ordinary skill in the art to produce said anti-coronavirus RBD antibodies.

The host cells used to express the anti-coronavirus RBD antibodies may be either a bacterial cell such as E.coli, yeast (e.g., S. cerevisiae), or a eukaryotic cell (e.g., a mammalian cell line). A mammalian cell of a well-defined type for this purpose, such as a myeloma cell, 3T3, HeLa, C6A2780, Vero, MOCK II, a Chinese hamster ovary (CHO), Sf9, Sf21, COS, NSO, or HEK293 cell line may be used.

The general methods by which the vectors may be constructed, transfection methods required to produce the host cell and culturing methods required to produce the antibodies, and fragments thereof, from said host cells all include conventional techniques. Although preferably the cell line used to produce the anti-coronavirus RBD antibodies is a mammalian cell line, any other suitable cell line, such as a bacterial cell line such as an E. coli-derived bacterial strain, or a yeast cell line, may be used.

Similarly, once produced the anti-coronavirus RBD antibodies may be purified according to standard procedures in the art, such as for example cross-flow filtration, ammonium sulphate precipitation, and affinity column chromatography.

### Diagnostic Uses of Anti-Coronavirus RBD Antibodies

Labeled antibodies described herein (including molecules comprising, or alternatively consisting of, antibody fragments or variants thereof) which specifically bind to a coronavirus RBD can be used for diagnostic purposes to detect, diagnose, prognose, or monitor diseases and/or disorders associated with the coronavirus infections. The invention provides for the detection of coronavirus infection comprising: (a) assaying the presence of a coronavirus RBD in a biological sample from a subject using one or more antibodies described herein that immunospecifically binds to a coronavirus RBD; and (b) comparing the level of a coronavirus RBD with a control, e.g., in normal biological samples, with no known coronavirus infection.

By “biological sample” is intended any fluids and/or cells obtained from a subject, body fluid, body tissue, body cell, cell line, tissue culture, or other source which may contain a coronavirus RBD protein or mRNA. Body fluids include, but are not limited to, sera, plasma, urine, synovial fluid, spinal fluid, saliva, and mucous. Tissues samples may be taken from virtually any tissue in the body. Tissue samples may also be obtained from autopsy material. Methods for obtaining tissue biopsies and body fluids from mammals are well known in the art. Where the biological sample is to include mRNA, a tissue biopsy is the preferred source.

In addition, the anti-coronavirus RBD antibodies described herein are useful in diagnostic assays for a coronavirus RBD expression in specific cells or tissues wherein the antibodies are labeled as described below and/or are immobilized on an insoluble matrix. Anti-coronavirus RBD antibodies also are useful for the affinity purification of a coronavirus RBD from recombinant cell culture or natural sources.

Anti-coronavirus RBD antibodies can be used for the detection of a coronavirus RBD in any one of a number of well-known diagnostic assay methods. For example, a biological sample may be assayed for coronavirus RBD by obtaining the sample from a desired source, admixing the sample with anti-coronavirus RBD antibody to allow the antibody to form antibody/coronavirus RBD complex with any coronavirus RBD present in the mixture, and detecting any antibody/coronavirus RBD complex present in the mixture. The biological sample may be prepared for assay by methods known in the art which are suitable for the particular sample. The methods of admixing the sample with antibodies and the methods of detecting antibody/coronavirus RBD complex are chosen according to the type of assay used. Such assays include competitive and sandwich assays, and steric inhibition assays. Competitive and sandwich methods employ a phase-separation step as an integral part of the method while steric inhibition assays are conducted in a single reaction mixture.

Analytical methods for coronavirus RBD all use one or more of the following reagents: labeled coronavirus RBD analogue, immobilized coronavirus RBD analogue, labeled anti-coronavirus RBD antibody, immobilized anti-coronavirus RBD antibody and steric conjugates. The labeled reagents also are known as “tracers.”

The label used is any detectable functionality that does not interfere with the binding of coronavirus RBD and anti-coronavirus RBD antibody. Numerous labels are known for use in immunoassay, examples including moieties that may be detected directly, such as fluorochrome, chemiluminescent, and radioactive labels, as well as moieties, such as enzymes, that must be reacted or derivatized to be detected. Examples of such labels include the radioisotopes ³²P ¹⁴C ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Conventional methods are available to bind these labels covalently to proteins or polypeptides. For instance, coupling agents such as dialdehydes, carboduimides, dimaleimides, bis-imidates, bis-diazotized benzidine, and the like may be used to tag the antibodies with the above-described fluorescent, chemiluminescent, and enzyme labels. See, for example, U.S. Pat. Nos. 3,940,475 (fluorimetry) and 3,645,090 (enzymes); Hunter et al. Nature, 144: 945 (1962); David et al. Biochemistry, 13: 1014-1021 (1974); Pain et al. J. Immunol. Methods, 40: 219-230 (1981); and Nygren, J. Histochem. and Cytochem., 30: 407-412 (1982). Preferred labels herein are enzymes such as horseradish peroxidase and alkaline phosphatase.

The conjugation of such label, including the enzymes, to the antibody is a standard manipulative procedure for one of ordinary skill in immunoassay techniques. See, for example, O′Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Immobilization of reagents is required for certain assay methods. Immobilization entails separating the anti-coronavirus RBD antibody from any coronavirus RBD that remains free in solution. This conventionally is accomplished by either insolubilizing the anti-coronavirus RBD antibody or coronavirus RBD analogue before the assay procedure, as by adsorption to a water-insoluble matrix or surface (Bennich et al. U.S. Pat. No. 3,720,760), by covalent coupling (for example, using glutaraldehyde cross-linking), or by insolubilizing the anti-coronavirus RBD antibody or coronavirus RBD analogue afterward, e.g., by immunoprecipitation.

Other assay methods, known as competitive or sandwich assays, are well established and widely used in the commercial diagnostics industry.

Competitive assays rely on the ability of a tracer coronavirus RBD analogue to compete with the test sample coronavirus RBD for a limited number of anti-coronavirus RBD antibody antigen-binding sites. The anti-coronavirus RBD antibody generally is insolubilized before or after the competition and then the tracer and coronavirus RBD bound to the anti-coronavirus RBD antibody are separated from the unbound tracer and coronavirus RBD. This separation is accomplished by decanting (where the binding partner was preinsolubilized) or by centrifuging (where the binding partner was precipitated after the competitive reaction). The amount of test sample coronavirus RBD is inversely proportional to the amount of bound tracer as measured by the amount of marker substance. Dose-response curves with known amounts of coronavirus RBD are prepared and compared with the test results to quantitatively determine the amount of coronavirus RBD present in the test sample. These assays are called ELISA systems when enzymes are used as the detectable markers.

Another species of competitive assay, called a “homogeneous” assay, does not require a phase separation. Here, a conjugate of an enzyme with the coronavirus RBD is prepared and used such that when anti-coronavirus RBD antibody binds to the coronavirus RBD the presence of the anti-coronavirus RBD antibody modifies the enzyme activity. In this case, the coronavirus RBD or its immunologically active fragments are conjugated with a bifunctional organic bridge to an enzyme such as peroxidase. Conjugates are selected for use with anti-coronavirus RBD antibody so that binding of the anti-coronavirus RBD antibody inhibits or potentiates the enzyme activity of the label. This method per se is widely practiced under the name of EMIT.

Steric conjugates are used in steric hindrance methods for homogeneous assay. These conjugates are synthesized by covalently linking a low-molecular-weight hapten to a small coronavirus RBD fragment so that antibody to hapten is substantially unable to bind the conjugate at the same time as anti-coronavirus RBD antibody. Under this assay procedure the coronavirus RBD present in the test sample will bind anti-coronavirus RBD antibody, thereby allowing anti-hapten to bind the conjugate, resulting in a change in the character of the conjugate hapten, e.g., a change in fluorescence when the hapten is a fluorophore.

Sandwich assays particularly are useful for the determination of coronavirus RBD or anti-coronavirus RBD antibodies. In sequential sandwich assays an immobilized anti-coronavirus RBD antibody is used to adsorb test sample coronavirus RBD, the test sample is removed as by washing, the bound coronavirus RBD is used to adsorb a second, labeled anti-coronavirus RBD antibody and bound material is then separated from residual tracer. The amount of bound tracer is directly proportional to test sample coronavirus RBD. In “simultaneous” sandwich assays the test sample is not separated before adding the labeled anti-coronavirus RBD. A sequential sandwich assay using an anti-coronavirus RBD monoclonal antibody as one antibody and a polyclonal anti-coronavirus RBD antibody as the other is useful in testing samples for coronavirus RBD.

The foregoing are merely exemplary diagnostic assays for coronavirus RBD. Other methods now or hereafter developed that use anti-coronavirus RBD antibody for the determination of coronavirus RBD are included within the scope hereof, including the bioassays described above.

### Therapeutic Methods

Antibodies described herein (including molecules comprising, or alternatively consisting of, antibody fragments or variants thereof) which specifically bind to coronavirus RBD can be used for diagnostic purposes to detect, diagnose, prognose, or monitor coronavirus infections, preferably SARS-CoV-2 infections, and conditions associated therewith.

### Therapeutic Compositions and Administration of Anti-Coronavirus RBD Antibodies

Therapeutic formulations of the anti-coronavirus RBD antibodies described herein are prepared for storage by mixing antibody having the desired degree of purity with optional physiologically acceptable carriers, excipients, or stabilizers (Remington: The Science and Practice of Pharmacy, 19th Edition, Alfonso, R., Ed, Mack Publishing Co. (Easton, Pa.: 1995)), in the form of lyophilized cake or aqueous solutions. Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The anti-coronavirus RBD antibody to be used for in vivo administration must be sterile. This is readily accomplished by filtration through sterile filtration membranes, prior to or following lyophilization and reconstitution. The anti-coronavirus RBD antibody may be stored in lyophilized form or in solution.

Therapeutic anti-coronavirus RBD antibody compositions generally are placed into a container having a sterile access port, for example, an intravenous solution bag or vial having a stopper pierceable by a hypodermic injection needle.

The route of anti-coronavirus RBD antibody administration is in accord with known methods, e.g. injection or infusion by intravenous, intraperitoneal, intracerebral, subcutaneous, intramuscular, intraocular, inhaled, optionally intransal, intrapulmonary, intraarterial, intracerebrospinal, or intralesional routes, or by sustained release systems as noted below. The anti-coronavirus RBD antibody may be administered as an aerosol, via intranasal and/or intrapulmonary routes. Preferably the antibody is given systemically.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman et al. Biopolymers, 22: 547-556 (1983)), poly (2-hydroxyethyl-methacrylate) (Langer et al. J. Biomed. Mater. Res., 15: 167-277 (1981) and Langer, Chem. Tech., 12: 98-105 (1982)), ethylene vinyl acetate (Langer et al. supra) or poly-D-(-)-3-hydroxybutyric acid (EP 133,988). Sustained-release anti-coronavirus RBD antibody compositions also include liposomally entrapped antibody. Liposomes containing antibody are prepared by methods known per se: DE 3,218,121; Epstein et al. Proc. Natl. Acad. Sci. USA, 82: 3688-3692 (1985); Hwang et al. Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamelar type in which the lipid content is greater than about 30 mol. % cholesterol, the selected proportion being adjusted for the optimal antibody therapy.

Anti-coronavirus RBD antibody can also be administered by inhalation. Commercially available nebulizers for liquid formulations, including jet nebulizers and ultrasonic nebulizers are useful for administration. Liquid formulations can be directly nebulized and lyophilized powder can be nebulized after reconstitution. Alternatively, anti-coronavirus RBD antibody can be aerosolized using a fluorocarbon formulation and a metered dose inhaler, or inhaled as a lyophilized and milled powder. The anti-coronavirus RBD antibodies described herein may be formulated to be administered via inhalation, for example, via intranasal and/or intrapulmonary routes.

For example, the anti-coronavirus RBD antibodies and compositions comprising the same described herein may be delivered in the form of vapors, drops, sprays, aerosols, or a powder. The anti-coronavirus RBD antibodies and compositions comprising the same may be delivered by a device configured for inhalation administration. The device configured for inhalation delivery of the antibody or antigen binding fragment or composition described herein. The device may be configured for inhalation delivery via intranasal, intrapulmonary, or a combination thereof.

The device may be an insufflator, breath actuated inhaler, mechanical powder sprayer, electrically power nebulizer (atomizer), nebulizer, atomizer, gas driven spray systems, gas driven atomixers, or mechanical pump sprays.

The inventors surprising discovered that the combination of an anti-coronavirus RBD IgA antibody delivered via inhalation was more effective in treating coronavirus infections.

An “effective amount” of anti-coronavirus RBD antibody to be employed therapeutically will depend, for example, upon the therapeutic objectives, the route of administration, the type of anti-coronavirus RBD antibody employed, and the condition of the patient. Accordingly, it will be necessary for the therapist to titer the dosage and modify the route of administration as required to obtain the optimal therapeutic effect. Typically, the clinician will administer the anti-coronavirus RBD antibody until a dosage is reached that achieves the desired effect. The progress of this therapy is easily monitored by conventional assays.

The patients to be treated with the anti-coronavirus RBD antibody described herein include preclinical patients or those with recent onset of immune-mediated disorders, and particularly autoimmune disorders. Patients are candidates for therapy in accord with this invention until such point as no healthy tissue remains to be protected from immune-mediated destruction. For example, a patient suffering from insulin-dependent diabetes mellitus (IDDM) can benefit from therapy with an anti-coronavirus RBD antibody described herein until the patient's pancreatic islet cells are no longer viable. It is desirable to administer an anti-coronavirus RBD antibody as early as possible in the development of the immune-mediated or autoimmune disorder, and to continue treatment for as long as is necessary for the protection of healthy tissue from destruction by the patient's immune system. For example, the IDDM patient is treated until insulin monitoring demonstrates adequate islet response and other indicia of islet necrosis diminish (e.g. reduction in anti-islet antibody titers), after which the patient can be withdrawn from anti-coronavirus RBD antibody treatment for a trial period during which insulin response and the level of anti-islet antibodies are monitored for relapse.

In the treatment, prevention, and/or amelioration of an immune-mediated or autoimmune disorder by an anti-coronavirus RBD antibody, the antibody composition comprising the anti-coronavirus RBD antibodies described herein may be formulated, dosed, and administered in a fashion consistent with good medical practice. Factors for consideration in this context include the particular disorder being treated, the particular mammal being treated, the clinical condition of the individual patient, the cause of the disorder, the site of delivery of the antibody, the particular type of antibody, the method of administration, the scheduling of administration, and other factors known to medical practitioners. The “therapeutically effective amount” of antibody to be administered will be governed by such considerations, and is the minimum amount necessary to prevent, ameliorate, or treat the disorder, including treating chronic autoimmune conditions and immunosuppression maintenance in transplant recipients. Such amount is preferably below the amount that is toxic to the host or renders the host significantly more susceptible to infections.

The anti-coronavirus RBD antibodies and antibody compositions described herein, may be used to treat, prevent, ameliorate, diagnose or prognose coronavirus infections, preferably SARS-CoV-2 infections, in mammals, preferably humans.

Therapeutic and pharmaceutical compositions comprising the anti-coronavirus RBD antibodies described herein may be used to treat, prevent, ameliorate, diagnose or prognose, coronavirus infection and/or medical conditions associated therewith (e.g. acute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof).

Therapeutic or pharmaceutical compositions comprising the anti-coronavirus RBD antibodies described herein may be administered to an animal to treat, prevent or ameliorate coronavirus infections and/or conditions associated with coronavirus infections. Examples of conditions associated with coronavirus infections include, but are not limited to, asthmaacute respiratory failure, pneumonia, acute respiratory distress syndrome (ARDS), acute liver injury, acute cardiac injury, secondary infection(s), acute kidney injury, septic shock, disseminated intravascular coagulation, blood clots, multisystem inflammatory syndrome, chronic fatigue, rhabdomyolysis, and combinations thereof.

As a general proposition, the initial pharmaceutically effective amount of the antibody administered parenterally will be in the range of about 0.1 to 50 mg/kg of patient body weight per day, with the typical initial range of antibody used being 0.3 to 20 mg/kg/day, more preferably 0.3 to 15 mg/kg/day. The desired dosage can be delivered by a single bolus administration, by multiple bolus administrations, or by continuous infusion administration of antibody, depending on the pattern of pharmacokinetic decay that the practitioner wishes to achieve.

The antibody need not be, but is optionally, formulated with one or more agents currently used to prevent or treat the coronavirus infection or condition associated with coronavirus infection in question. The effective amount of such other agents depends on the amount of anti-coronavirus RBD antibody present in the formulation, the type of disorder or treatment, and other factors discussed above. These are generally used in the same dosages and with administration routes as used hereinbefore or about from 1 to 99% of the heretofore employed dosages.

### Kits

A pharmaceutical pack or kit may comprise one or more containers filled with one or more of the ingredients of the pharmaceutical compositions comprising the anti-coronavirus RBD antibodies described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

Kits that can be used in the methods described herein. A kit may comprise an antibody described herein, preferably a purified antibody, in one or more containers. In an alternative embodiment, a kit comprises an antibody fragment that immunospecifically binds to coronavirus RBD. Kits may contain a substantially isolated coronavirus RBD polypeptide as a control.

Kits may further comprise a control antibody which does not react with coronavirus RBD. In another specific embodiment, the kits of the present invention contain a means for detecting the binding of an antibody to a coronavirus RBD (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In an embodiments, the kit may include a recombinantly produced or chemically synthesized a coronavirus RBD. The coronavirus RBD provided in the kit may also be attached to a solid support. In another embodiment, the detecting means of the above-described kit includes a solid support to which coronavirus RBD is attached. Such a kit may also include a non-attached reporter-labeled anti-human antibody. In this embodiment, binding of the antibody to a coronavirus RBD can be detected by binding of the said reporter-labeled antibody.

A diagnostic kit for use in screening a biological sample containing antigens of the polypeptide described herein. The diagnostic kit includes a substantially isolated antibody specifically immunoreactive with a coronavirus RBD, and means for detecting the binding of a coronavirus RBD to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, a biological sample is reacted with a solid phase reagent having a surface-bound coronavirus RBD obtained by the methods of the present invention. After coronavirus RBD binds to a specific antibody, the unbound serum components are removed by washing, reporter-labeled anti-human antibody is added, unbound anti-human antibody is removed by washing, and a reagent is reacted with reporter-labeled anti-human antibody to bind reporter to the reagent in proportion to the amount of bound anti-coronavirus RBD antibody on the solid support. Typically, the reporter is an enzyme which is detected by incubating the solid phase in the presence of a suitable fluorometric, luminescent or colorimetric substrate.

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant coronavirus RBD, and a reporter-labeled anti-human antibody for detecting surface-bound anti-coronavirus RBD antibody.

Further details described herein can be found in the following example, which further defines the scope described herein. All references cited throughout the specification, and the references cited therein, are hereby expressly incorporated by reference in their entirety.

### EXAMPLE 1

### Anti-SARS-CoV-2 Receptor Binding Domain Monoclonal IgA

A custom anti-SARS-CoV-2 receptor binding domain monoclonal IgA antibody was developed described herein. This antibody was tested in vitro utilizing an enzyme-linked immunosorbent assay and showed that it bound strongly to both an isolated receptor binding domain (RBD) construct and a full spike ectodomain trimer suggesting it has the ability to bind these domains on the SARS-CoV-2 virus (FIG. 3). The IC50 for the ELISA was 0.0006941 mg/ml wherein seronegative blood serum was spiked with recombinant IgA, then added to our ELISA at a 1:400 dilution of serum into sample buffer. Accounting for this dilution the expected IC50 in the assay is 1.73525 ng/ml. This data suggests that the anti-SARS-CoV-2 receptor binding domain monoclonal IgA antibody has strong potential as a coronavirus neutralizing antibody.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. An isolated anti-coronavirus receptor binding domain (RBD) antibody or an antigen binding fragment thereof comprising a heavy chain variable (VH) region and a light chain variable (VL) region that specifically binds a coronavirus receptor binding domain polypeptide, wherein:
the light chain variable region comprises an amino acid sequence that shares at least about 80% sequence homology to the amino acid sequence of SEQ ID NO: 23; and
the heavy chain variable region comprises an amino acid sequence that shares at least about 80% sequence homology to the amino acid sequence of SEQ ID NO: 24.

2. The antibody or antigen binding fragment thereof of claim 1, wherein the heavy chain constant region comprises an amino acid sequence that shares at least about 80% sequence homology to the amino acid sequence of SEQ ID NO: 5.

3. The antibody or antigen binding fragment thereof of claim 1, wherein the light chain constant region comprises an amino acid sequence that shares at least about 80% sequence homology to the amino acid sequence of SEQ ID NO: 6.

4-5. (canceled)

6. The antibody or antigen binding fragment thereof of claim 1, wherein the light chain variable region comprises CDR1 comprising an amino acid sequence of SEQ ID NO: 10, CDR2 comprising an amino acid sequence of SEQ ID NO: 12, and CDR3 comprising an amino acid sequence of SEQ ID NO: 14.

7. The antibody or antigen binding fragment thereof of claim 1, wherein the heavy chain variable region comprises CDR1 comprising an amino acid sequence of SEQ ID NO: 17, CDR2 comprising an amino acid sequence of SEQ ID NO: 19, and CDR3 comprising an amino acid sequence of SEQ ID NO: 21.

8. The antibody or antigen binding fragment thereof of claim 6, wherein the light chain variable region comprises framework sections comprising the amino acid sequences of SEQ ID NOs: 9, 11, 13, and 15.

9. The antibody or antigen binding fragment thereof of claim 7, wherein the heavy chain variable region comprises framework sections comprising the amino acid sequences of SEQ ID NOs: 16, 18, 20, and 22.

10. The antibody or antigen binding fragment thereof of claim 1, wherein the antibody specifically binds to the coronavirus RBD comprising the amino acid sequence of SEQ ID NO: 1, 2, 3, or 4, or a combination thereof.

11-12. (canceled)

13. The antibody or antigen binding fragment thereof of claim 1, wherein the antigen-binding fragment is an Fab, Fab′, or F(ab′)2.

14. The antibody or antigen binding fragment thereof of claim 1, wherein the antibody is an IgG, IgM, IgE, IgD, or IgA antibody.

15-16. (canceled)

17. The antibody or antigen binding fragment thereof of claim 1, wherein the antibody is monoclonal.

18. (canceled)

19. The antibody or antigen binding fragment thereof of claim 1, wherein the antibody is a chimeric antibody.

20. The antibody or antigen binding fragment thereof of claim 1, wherein the antibody or antigen-binding fragment thereof, is conjugated to a label, cytotoxic agent, immunosuppressive agent.

21-22. (canceled)

23. The antibody or antigen binding fragment thereof of claim 1, wherein the coronavirus is SARS-CoV-1, MERS-CoV, SARS-CoV-2 (COVID-19), HCoV-0C43, HCoV-HKU1, HCoV-NL63, or HCoV-229E, or a combination thereof.

24. (canceled)

25. The antibody or antigen binding fragment thereof of claim 1, wherein the antibody selectively binds to an epitope contained within amino acids 331-524 of the Spike protein of SARS-CoV-2 (SEQ ID NO: 1), amino acid residues 318-510 of the Spike protein of SARS-CoV (SEQ ID NO: 2), amino acid residues 377-588 of the MERS-CoV spike protein (SEQ ID NO: 3), or amino acids 319-541 of the SARS CoV-2 spike receptor-binding domain (SEQ ID NO: 4), or a combination thereof.

26. (canceled)

27. A composition comprising the antibody or antigen binding fragment thereof of claim 1.

28. The composition of claim 27, wherein the composition is a pharmaceutical composition further comprising a pharmaceutical excipient, a carrier, a diluent, or an adjuvant, or a combination thereof.

29. (canceled)

30. An isolated nucleotide comprising a nucleic acid sequence comprising at least about 80% sequence homology with a nucleic acid sequence that encodes the amino acid sequence of SEQ ID NO: 5, 6, 7, 8, 9, 10, 11, 12, 13, 14, 15, 16, 17, 18, 19, 21, 22, 23, or 24.

31. The isolated nucleotide of claim 30, wherein the sequence homology is at least about 95% sequence homology.

32. A vector comprising the isolated nucleotide of claim 30.

33. A host cell comprising the vector of claim 32.

34-37. (canceled)

38. A method for detecting a coronavirus receptor binding domain polypeptide comprising contacting a sample with the antibody or antigen binding fragment thereof of claim 1.

39. The method of claim 38, wherein the antibody, or antigen binding fragment thereof, attached to a solid phase support.

40-44. (canceled)

45. A method for treating a viral infection comprising administering an effective amount of the antibody or antigen binding fragment thereof of claim 1.

46. The method of claim 45, wherein the viral infection is coronavirus virus infection.

47. The method of claim 46, wherein the coronavirus is SARS-CoV-1, MERS-CoV, SARS-CoV-2 (COVID-19), HCoV-0C43, HCoV-HKU1, HCoV-NL63, or HCoV-229E, or a combination thereof.

48-53. (canceled)

54. The method of claim 45, wherein the antibody or antigen binding fragment thereof is administered intravenously, inhalation, subcutaneously, via infusion, orally, intrathecally, or intraperitoneally, parenterally, or a combination thereof.

55-59. (canceled)

60. A kit comprising the antibody or antigen binding fragment thereof of claim 1.

61-63. (canceled)

64. A device configured for inhalation delivery comprising the antibody or antigen binding fragment of claim 1.

65-66. (canceled)

67. The device of claim 64, wherein the device is an insufflator, breath actuated inhaler, mechanical powder sprayer, electrically power nebulizer (atomizer), nebulizer, atomizer, gas driven spray systems, gas driven atomixers, or mechanical pump spray.

68-90. (canceled)


SEQUENCE LISTING (24 sequences)

SEQ ID NO: 1 - protein, 194 aa, Artificial Sequence
  spike glycoprotein [Severe acute respiratory syndrome coronavirus 2]
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 2 - protein, 193 aa, Artificial Sequence
  spike glycoprotein S [SARS coronavirus BJ01]
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATV

SEQ ID NO: 3 - protein, 212 aa, Artificial Sequence
  spike glycoprotein [MERS coronavirus]
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 4 - protein, 223 aa, Artificial Sequence
  SARS CoV-2 spike receptor-binding domain
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 5 - protein, 353 aa, Artificial Sequence
  human IgA heavy chain (IGHA1) constant region
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 6 - protein, 106 aa, Artificial Sequence
  human IgA light chain (IGKA1) constant region
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 214 aa, Artificial Sequence
  Anti-coronavirus RBD Ab - Light Chain
DILLTQSPAILSVSPGERVSFSCRASQSIGTSIHWYHQRTNGSPRLLIKYASESISGIPSRFSGSGSGTDFTLSINSVESEDIADYYCQQSNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 471 aa, Artificial Sequence
  Anti-coronavirus RBD Ab - Heavy Chain
EVLLQQSGPELVKPGASVKIPCKASGYTFTDYNMDWVKQSHGKSLEWIGDINPNNGFTIYNQKFKGKATLTVDKSSSTAYMELRSLTSEDTAVYYCAREGYGNYFDYWGQGTTLTVSSASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 9 - protein, 23 aa, Artificial Sequence
  Anti-coronavirus RBD Ab - Light Chain FR1
DILLTQSPAILSVSPGERVSFSC

SEQ ID NO: 10 - protein, 11 aa, Artificial Sequence
  Anti-coronavirus RBD Ab - Light Chain CDR1
RASQSIGTSIH

SEQ ID NO: 11 - protein, 15 aa, Artificial Sequence
  Anti-coronavirus RBD Ab - Light Chain FR2
WYHQRTNGSPRLLIK

SEQ ID NO: 12 - protein, 7 aa, Artificial Sequence
  Anti-coronavirus RBD Ab - Light Chain CDR2
YASESIS

SEQ ID NO: 13 - protein, 32 aa, Artificial Sequence
  Anti-coronavirus RBD Ab - Light Chain FR3
GIPSRFSGSGSGTDFTLSINSVESEDIADYYC

SEQ ID NO: 14 - protein, 9 aa, Artificial Sequence
  Anti-coronavirus RBD Ab - Light Chain CDR3
QQSNNWPTT

SEQ ID NO: 15 - protein, 10 aa, Artificial Sequence
  Anti-coronavirus RBD Ab - Light Chain FR4
FGAGTKLELK

SEQ ID NO: 16 - protein, 25 aa, Artificial Sequence
  Anti-coronavirus RBD Ab - Heavy Chain FR1
EVLLQQSGPELVKPGASVKIPCKAS

SEQ ID NO: 17 - protein, 7 aa, Artificial Sequence
  Anti-coronavirus RBD Ab - Heavy Chain CDR1
GYTFTDY

SEQ ID NO: 18 - protein, 19 aa, Artificial Sequence
  Anti-coronavirus RBD Ab - Heavy Chain FR2
NMDWVKQSHGKSLEWIGDI

SEQ ID NO: 19 - protein, 6 aa, Artificial Sequence
  Anti-Coronavirus RBD Ab - Heavy Chain CDR2
NPNNGF

SEQ ID NO: 20 - protein, 41 aa, Artificial Sequence
  Anti-Coronavirus RBD Ab - Heavy Chain FR3
TIYNQKFKGKATLTVDKSSSTAYMELRSLTSEDTAVYYCAR

SEQ ID NO: 21 - protein, 9 aa, Artificial Sequence
  Anti-Coronavirus RBD Ab - Heavy Chain CDR3
EGYGNYFDY

SEQ ID NO: 22 - protein, 11 aa, Artificial Sequence
  Anti-Coronavirus RBD Ab - Heavy Chain FR4
WGQGTTLTVSS

SEQ ID NO: 23 - protein, 107 aa, Artificial Sequence
  Anti-Coronavirus RBD Ab - Variable Light Domain
DILLTQSPAILSVSPGERVSFSCRASQSIGTSIHWYHQRTNGSPRLLIKYASESISGIPSRFSGSGSGTDFTLSINSVESEDIADYYCQQSNNWPTTFGAGTKLELK

SEQ ID NO: 24 - protein, 118 aa, Artificial Sequence
  Anti-Coronavirus RBD Ab - Variable Heavy Domain
EVLLQQSGPELVKPGASVKIPCKASGYTFTDYNMDWVKQSHGKSLEWIGDINPNNGFTIYNQKFKGKATLTVDKSSSTAYMELRSLTSEDTAVYYCAREGYGNYFDYWGQGTTLTVSS